(19) United States
(12) Patent Application Publication
(10) Pub. No.: US 2025/0335825 A1
(43) Pub. Date: Oct. 30, 2025

(54) DATA AUGMENTATION AND ENCODING OF MULTI-CHAIN PROTEIN STRUCTURES

(71) Applicant: Amgen Inc., Thousand Oaks, CA (US)

(72) Inventors: Christopher Langmead, Monroeville, PA (US); Mani Jain, Bellevue, WA (US); Huimin Zhuge, San Jose, CA (US)

(73) Assignee: Amgen Inc., Thousand Oaks, CA (US)

(21) Appl. No.: 19/188,772

(22) Filed: Apr. 24, 2025

Related U.S. Application Data
(60) Provisional application No. 63/638,366, filed on Apr. 24, 2024.

(51) Int. Cl. G06N 20/00

(52) U.S. Cl. CPC G06N 20/00

(57) ABSTRACT

Described herein are techniques for predicting one or more properties of a multi-chain protein, the multi-chain protein including at least a first chain and a second chain. In some embodiments, the techniques include: obtaining sequence data for the multi-chain protein, the sequence data indicating a first amino acid sequence specifying at least a portion of the first chain and a second amino acid sequence specifying at least a portion of the second chain; generating a concatenated amino acid sequence by concatenating the first amino acid sequence, a linker, and the second amino acid sequence; encoding the concatenated amino acid sequence to obtain a numeric representation of the concatenated amino acid sequence; and processing the numeric representation of the concatenated amino acid sequence using a trained machine learning model to obtain an output indicative of the one or more properties of the multi-chain protein.

### CROSS-REFERENCE TO RELATED APPLICATIONS

The present application claims the benefit of priority under 35 U.S.C. § 119 (e) of U.S. Provisional Patent Application Ser. No. 63/638,366 filed on Apr. 24, 2024, under Attorney Docket No. A1350.70007US00, and entitled “DATA AUGMENTATION AND ENCODING OF MULTI-CHAIN PROTEINS,” which is incorporated by reference herein in its entirety.

### BACKGROUND

A “multi-chain protein” or “protein complex” refers to a group of two or more associated polypeptide chains. A “polypeptide chain” generally refers to a linear, unbranched, series of amino acids linked to one another by peptide bonds.

Proteins can be developed for a variety of different applications such as, for example, protein-based therapeutics. One or more properties of a protein can inform whether the protein will be suitable for a particular application. However, because a variety of different factors impact the properties of a particular protein, it can be challenging and complex to optimize a protein for said properties.

### SUMMARY

Some aspects provide for a method of predicting one or more properties of a multi-chain protein, the multi-chain protein including at least a first chain and a second chain. In some embodiments, the method comprises: using at least one computer hardware processor to perform: obtaining sequence data for the multi-chain protein, the sequence data indicating a first amino acid sequence specifying at least a portion of the first chain and a second amino acid sequence specifying at least a portion of the second chain; generating a concatenated amino acid sequence by concatenating the first amino acid sequence, a linker, and the second amino acid sequence; encoding the concatenated amino acid sequence to obtain a numeric representation of the concatenated amino acid sequence; and processing the numeric representation of the concatenated amino acid sequence using a trained machine learning model to obtain an output indicative of the one or more properties of the multi-chain protein.

Some aspects provide for a system, comprising: at least one computer hardware processor; and at least one non-transitory computer-readable storage medium storing processor-executable instructions that, when executed by the at least one computer hardware processor, cause the at least one computer hardware processor to perform a method of predicting one or more properties of a multi-chain protein, the multi-chain protein including at least a first chain and a second chain. In some embodiments, the method comprises: obtaining sequence data for the multi-chain protein, the sequence data indicating a first amino acid sequence specifying at least a portion of the first chain and a second amino acid sequence specifying at least a portion of the second chain; generating a concatenated amino acid sequence by concatenating the first amino acid sequence, a linker, and the second amino acid sequence; encoding the concatenated amino acid sequence to obtain a numeric representation of the concatenated amino acid sequence; and processing the numeric representation of the concatenated amino acid sequence using a trained machine learning model to obtain an output indicative of the one or more properties of the multi-chain protein.

Some aspects provide for at least one non-transitory computer-readable storage medium storing processor-executable instructions that, when executed by at least one computer hardware processor, cause the at least one computer hardware processor to perform a method of predicting one or more properties of a multi-chain protein, the multi-chain protein including at least a first chain and a second chain. In some embodiments, the method comprises: obtaining sequence data for the multi-chain protein, the sequence data indicating a first amino acid sequence specifying at least a portion of the first chain and a second amino acid sequence specifying at least a portion of the second chain; generating a concatenated amino acid sequence by concatenating the first amino acid sequence, a linker, and the second amino acid sequence; encoding the concatenated amino acid sequence to obtain a numeric representation of the concatenated amino acid sequence; and processing the numeric representation of the concatenated amino acid sequence using a trained machine learning model to obtain an output indicative of the one or more properties of the multi-chain protein.

Some aspects provide for a method for training a machine learning model to predict one or more properties of a multi-chain protein. In some embodiments, the method comprises: using at least one computer hardware processor to perform: generating training data at least in part by: obtaining initial data for a plurality of multi-chain proteins, each of the plurality of multi-chain proteins including at least two chains, wherein the initial data indicates, for each particular multi-chain protein of the plurality of multi-chain proteins, one or more properties of the particular multi-chain protein and sequence data for the particular multi-chain protein that indicates a respective amino acid sequence for each of the at least two chains of the particular multi-chain protein; augmenting the initial data to obtain augmented data, the augmenting comprising, for each particular multi-chain protein of the plurality of multi-chain proteins, generating a respective concatenated amino acid sequence for the particular multi-chain protein by concatenating a linker and the respective amino acid sequences indicated for the at least two chains of the particular multi-chain protein; and encoding the augmented data to obtain the training data; training the machine learning model using the generated training data to predict the one or more properties of the multi-chain protein thereby obtaining values for parameters of the trained machine learning model; and storing the parameter values for the trained machine learning model.

Some aspects provide for a system, comprising: at least one computer hardware processor; and at least one non-transitory computer-readable storage medium storing processor-executable instructions that, when executed by the at least one computer hardware processor, causes the at least one computer hardware processor to perform a method for training a machine learning model to predict one or more properties of a multi-chain protein. In some embodiments, the method comprises: generating training data at least in part by: obtaining initial data for a plurality of multi-chain proteins, each of the plurality of multi-chain proteins including at least two chains, wherein the initial data indicates, for each particular multi-chain protein of the plurality of multi-chain proteins, one or more properties of the particular multi-chain protein and sequence data for the particular multi-chain protein that indicates a respective amino acid sequence for each of the at least two chains of the particular multi-chain protein; augmenting the initial data to obtain augmented data, the augmenting comprising, for each particular multi-chain protein of the plurality of multi-chain proteins, generating a respective concatenated amino acid sequence for the particular multi-chain protein by concatenating a linker and the respective amino acid sequences indicated for the at least two chains of the particular multi-chain protein; and encoding the augmented data to obtain the training data; training the machine learning model using the generated training data to predict the one or more properties of the multi-chain protein thereby obtaining values for parameters of the trained machine learning model; and storing the parameter values for the trained machine learning model.

Some aspects provide for at least one non-transitory computer-readable storage medium storing processor-executable instructions that, when executed by at least one computer hardware processor, cause the at least one computer hardware processor to perform a method for training a machine learning model to predict one or more properties of a multi-chain protein. In some embodiments, the method comprises: generating training data at least in part by: obtaining initial data for a plurality of multi-chain proteins, each of the plurality of multi-chain proteins including at least two chains, wherein the initial data indicates, for each particular multi-chain protein of the plurality of multi-chain proteins, one or more properties of the particular multi-chain protein and sequence data for the particular multi-chain protein that indicates a respective amino acid sequence for each of the at least two chains of the particular multi-chain protein; augmenting the initial data to obtain augmented data, the augmenting comprising, for each particular multi-chain protein of the plurality of multi-chain proteins, generating a respective concatenated amino acid sequence for the particular multi-chain protein by concatenating a linker and the respective amino acid sequences indicated for the at least two chains of the particular multi-chain protein; and encoding the augmented data to obtain the training data; training the machine learning model using the generated training data to predict the one or more properties of the multi-chain protein thereby obtaining values for parameters of the trained machine learning model; and storing the parameter values for the trained machine learning model.

Some aspects provide for a method of predicting one or more properties of a multi-chain protein, the multi-chain protein including at least a first chain and a second chain. In some embodiments, the method comprises: using at least one computer hardware processor to perform: obtaining sequence data for the multi-chain protein, the sequence data indicating a first amino acid sequence specifying at least a portion of the first chain and a second amino acid sequence specifying at least a portion of the second chain; generating a concatenated amino acid sequence by concatenating the first amino acid sequence and the second amino acid sequence; encoding the concatenated amino acid sequence to obtain a numeric representation of the concatenated amino acid sequence; and processing the numeric representation of the concatenated amino acid sequence using a trained machine learning model to obtain an output indicative of the one or more properties of the multi-chain protein, wherein the trained machine learning model was trained at least in part by: generating training data at least in part by: obtaining initial data for a plurality of multi-chain proteins, each of the plurality of multi-chain proteins including at least two chains, wherein the initial data indicates, for each particular multi-chain protein of the plurality of multi-chain proteins, one or more properties of the particular multi-chain protein and sequence data for the particular multi-chain protein that indicates a respective amino acid sequence for each of the at least two chains of the particular multi-chain protein; augmenting the initial data to obtain augmented data, the augmenting comprising, for each particular multi-chain protein of the plurality of multi-chain proteins, generating a respective concatenated amino acid sequence for the particular multi-chain protein by concatenating a linker and the respective amino acid sequences indicated for the at least two chains of the particular multi-chain protein; and encoding the augmented data to obtain the training data; training the machine learning model using the generated training data to predict the one or more properties of the multi-chain protein thereby obtaining values for parameters of the trained machine learning model; and storing the parameter values for the trained machine learning model.

Embodiments of any of the above aspects may have one or more of the following features.

In some embodiments, processing the numeric representation of the concatenated amino acid sequence using the trained machine learning model to obtain the output indicative of the one or more properties of the multi-chain protein comprises processing the numeric representation of the concatenated amino acid sequence using the trained machine learning model to obtain an output indicative of a degree of aggregation.

In some embodiments, processing the numeric representation of the concatenated amino acid sequence using the trained machine learning model to obtain the output indicative of the one or more properties of the multi-chain protein comprises processing the numeric representation of the concatenated amino acid sequence using the trained machine learning model to obtain an output indicative of a viscosity of the multi-chain protein.

In some embodiments, processing the numeric representation of the concatenated amino acid sequence using the trained machine learning model to obtain the output indicative of the one or more properties of the multi-chain protein comprises processing the numeric representation of the concatenated amino acid sequence using the trained machine learning model to obtain an output indicative of a degree of stability of the multi-chain protein.

In some embodiments, processing the numeric representation of the concatenated amino acid sequence using the trained machine learning model to obtain the output indicative of the one or more properties of the multi-chain protein comprises processing the numeric representation of the concatenated amino acid sequence using the trained machine learning model to obtain an output indicative of a degree of bioavailability of the multi-chain protein.

In some embodiments, processing the numeric representation of the concatenated amino acid sequence using the trained machine learning model to obtain the output indicative of the one or more properties of the multi-chain protein comprises processing the numeric representation of the concatenated amino acid sequence using the trained machine learning model to obtain an output indicative of a degree of pharmacokinetic clearance of the multi-chain protein.

In some embodiments, processing the numeric representation of the concatenated amino acid sequence using the trained machine learning model to obtain the output indicative of the one or more properties of the multi-chain protein comprises processing the numeric representation of the concatenated amino acid sequence using the trained machine learning model to obtain an output indicative of a productivity of the multi-chain protein.

In some embodiments, processing the numeric representation of the concatenated amino acid sequence using the trained machine learning model to obtain the output indicative of the one or more properties of the multi-chain protein comprises processing the numeric representation of the concatenated amino acid sequence using the trained machine learning model to obtain an output indicative of a binding affinity of the multi-chain protein to a target.

Some embodiments further comprise reducing a dimensionality of the numeric representation of the concatenated amino acid sequence to obtain a reduced-dimension representation of the numeric representation, the reduced-dimension representation of the numeric representation having fewer dimensions than the numeric representation, wherein processing the numeric representation using the trained machine learning model to obtain the output indicative of the one or more properties of the multi-chain protein comprises processing the reduced-dimension representation of the numeric representation using the trained machine learning model to obtain the output indicative of the one or more properties of the multi-chain protein.

In some embodiments, reducing the dimensionality of the numeric representation of the concatenated amino acid sequence comprises reducing the dimensionality of the numeric representation of the concatenated amino acid sequence using principal components analysis (PCA).

In some embodiments, encoding the concatenated amino acid sequence to obtain the numeric representation of the concatenated amino acid sequence comprises encoding the concatenated amino acid sequence using a protein language model.

In some embodiments, the protein language model comprises an ESM-1b model.

In some embodiments, the linker comprises one or more mask tokens.

In some embodiments, the linker is a poly-alanine linker.

In some embodiments, the trained machine learning model was trained at least in part by: generating training data at least in part by: obtaining initial data for a plurality of multi-chain proteins, each of the plurality of multi-chain proteins including at least two chains, wherein the initial data indicates, for each particular multi-chain protein of the plurality of multi-chain proteins, one or more properties of the particular multi-chain protein and sequence data that indicates a respective amino acid sequence for each of the at least two chains of the particular multi-chain protein; augmenting the initial data to obtain augmented data, the augmenting comprising, for each particular multi-chain protein of the plurality of multi-chain proteins (i) generating a respective concatenated amino acid sequence for the particular multi-chain protein at least in part by concatenating a linker and the respective amino acid sequences indicated for the at least two chains of the particular multi-chain protein and/or (ii) generating permutations of respective amino acid sequences indicated for the at least two chains of the particular multi-chain protein; and encoding the augmented data to obtain the training data; training the machine learning model using the generated training data to predict the one or more properties of the multi-chain protein thereby obtaining values for parameters of the trained machine learning model; and storing the parameter values for the trained machine learning model.

In some embodiments, processing the numeric representation of the concatenated amino acid sequence using the trained machine learning model to obtain the output indicative of the one or more properties of the multi-chain protein comprises processing the numeric representation of the concatenated amino acid sequence using a non-linear regression model.

In some embodiments, the non-linear regression model is a logistic regression model.

Some embodiments further comprise: modifying, based on the output indicative of the one or more properties of the multi-chain protein, one or more residues of the first amino acid sequence and/or one or more residues of the second amino acid sequence.

Some embodiments further comprise: expressing, based on the output indicative of the one or more properties of the multi-chain protein, the multi-chain protein or a fragment of the multi-chain protein to confirm if the multi-chain protein has the one or more properties by performing an assay; and selecting, if results of the assay confirm the multi-chain protein has the one or more properties, the multi-chain protein for additional testing as a potential therapy.

In some embodiments, augmenting the initial data to obtain the augmented data further comprises, for each particular multi-chain protein of the plurality of multi-chain proteins, generating permutations of the respective amino acid sequences indicated for the at least two chains of the particular multi-chain protein.

In some embodiments, generating the permutations of the respective amino acid sequences comprises: arranging the respective amino acid sequences in a first order to obtain a first permutation of the respective amino acid sequences; and arranging the respective amino acid sequences in a second order to obtain a second permutation of the respective amino acid sequences, the second order being different from the first order.

In some embodiments, the linker comprises one or more mask tokens.

In some embodiments, encoding the augmented data to obtain the training data comprises encoding the augmented data using a protein language model.

In some embodiments, the protein language model is an ESM-1b model.

In some embodiments, encoding the augmented data comprises encoding the augmented data to obtain numeric representations of the respective concatenated amino acid sequences generated for the plurality of multi-chain proteins, and wherein generating the training data further comprises reducing a dimensionality of each of the numeric representations to obtain the training data.

In some embodiments, reducing the dimensionality of each of the numeric representations of the respective concatenated amino acid sequences generated for the plurality of multi-chain proteins comprises reducing the dimensionality of each of the numeric representations using principal components analysis (PCA).

In some embodiments, training the machine learning model to predict the one or more properties of the multi-chain protein comprises training the machine learning model to predict a degree to which the multi-chain protein will aggregate with at least one other protein.

In some embodiments, training the machine learning model to predict the one or more properties of the multi-chain protein comprises training the machine learning model to predict a viscosity of the multi-chain protein.

In some embodiments, the machine learning model is a generalized linear model.

In some embodiments, the generalized linear model is a logistic regression model.

### BRIEF DESCRIPTION OF DRAWINGS

Various aspects and embodiments of the disclosure provided herein are described below with reference to the following figures. The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1A and FIG. 1B are diagrams of illustrative techniques for predicting one or more properties of a multi-chain protein, according to some embodiments of the technology described herein.

FIG. 1C is a diagram of an illustrative technique for training a machine learning model to predict one or more properties of a multi-chain protein, according to some embodiments of the technology described herein.

FIG. 1D is a diagram of an illustrative technique for generating training data for training a machine learning model to predict one or more properties of a multi-chain protein, according to some embodiments of the technology described herein.

FIG. 2 is a block diagram of an example system 200 for predicting one or more properties of a multi-chain protein, according to some embodiments of the technology described herein.

FIG. 3 is a flowchart of an illustrative process 300 for predicting one or more properties of a multi-chain protein, according to some embodiments of the technology described herein.

FIG. 4 is a flowchart of an illustrative process 400 for training a machine learning model to predict one or more properties of a multi-chain protein, according to some embodiments of the technology described herein.

FIG. 5A, FIG. 5B, and FIG. 5C are illustrative examples of generating training data used for training a machine learning model to predict one or more properties of a multi-chain protein, according to some embodiments of the technology described herein.

FIG. 6 is a graph showing the accuracy of a machine learning model trained, according to embodiments of the technology described herein, to predict aggregation of multi-chain proteins.

FIG. 7 is a schematic diagram of an illustrative computing device with which aspects described herein may be implemented.

### DETAILED DESCRIPTION

Advancements in protein engineering technologies have enabled the development of proteins for a variety of different applications including, for example, protein-based therapeutics. Typically, when developing a protein for a particular application, one or more properties of the protein are used to gauge whether the protein is suitable for the application. Different properties of a protein may be indicative of its stability under certain environmental conditions, its deliverability (e.g., into the body, into the cell, etc.), its pharmacokinetics, and/or its pharmacodynamics, among other characteristics. Aggregation and viscosity are two examples of properties that may be indicative of its suitability for use in certain applications such as protein-based therapeutics, for example.

Various factors such as the particular amino acid sequence and/or the resulting structure of a protein may impact its properties. Due to the variety of factors, optimizing a protein for one or more properties is typically a complex process. One approach involves experimentally screening protein candidates for the desired property. However, this approach is resource intensive, time consuming, and expensive because it requires producing and performing experiments on each protein in a large set of candidate proteins being screened to determine which proteins have the desired properties.

Computational techniques have been developed to improve the efficiency of optimizing proteins for one or more properties. The techniques involve using computational models to predict properties of candidate proteins. The inputs to these models are encodings (e.g., numeric representations) of the primary sequence(s) of the protein. The inventors have recognized that a problem with conventional techniques is that they are sensitive to how multi-chain proteins are encoded and presented to the computational models, rendering such techniques inconsistent and unreliable. Multiple aspects of the conventional techniques contribute to their sensitivity.

First, when encoding the sequences of a multi-chain protein, the conventional techniques do not distinguish between sequences specifying different chains of the multi-chain protein. Rather, sequences specifying different chains of the multi-chain protein are concatenated, and the concatenated sequence is encoded to obtain a representation of the concatenated sequence, which is provided as input to the computational model. Because there is no distinction between the chains, the computational model incorrectly conceives the multi-chain protein as a single chain thereby decreasing the accuracy of the resulting predictions.

Second, the inventors have recognized that the order of sequences specifying chains of a multi-chain protein impacts the encoding of the multi-chain protein. Consider, for example, a multi-chain protein having a first chain specified by a first sequence and a second chain specified by a second sequence. Encoding the first sequence followed by the second sequence will result in a different numeric representation than the numeric representation resulting from encoding the second sequence followed by the first sequence. The conventional computational models are trained on a single permutation (e.g., order) of the sequences specifying the chains of each multi-chain protein (e.g., either (a) the first sequence followed by the second sequence, or (b) the second sequence followed by the first sequence). As a result, even if the model has been trained to predict the property of a particular multi-chain protein based on one permutation, it is not equipped to accurately predict the property of that particular multi-chain protein if a different permutation of the sequences specifying the chains of the multi-chain protein is encoded and provided as input to the trained model.

The inventors have further recognized that, due to the inefficiencies of experimentally determining the properties of a protein, it is both challenging and expensive to obtain the amount of data that is necessary for training a computational model to accurately predict a property of a protein.

Accordingly, the inventors have developed techniques that address the above-described shortcomings of the conventional techniques for predicting one or more properties of a multi-chain protein. In some embodiments, the techniques include: (a) obtaining, for the multi-chain protein, sequence data including amino acid sequences specifying at least a portion of each of the chains of the multi-chain protein, (b) generating a concatenated amino acid sequence by concatenating the amino acid sequences and one or more linkers (e.g., amino acid sequence, one or more tokens), (c) encoding the concatenated amino acid sequence (e.g., using a protein language model) to obtain a numeric representation of the concatenated amino acid sequence, and (d) processing the numeric representation of the concatenated amino acid sequence using a trained machine learning model to obtain an output indicative of the one or more properties of the multi-chain protein.

Including a linker in the concatenated amino acid sequence serves to distinguish between the amino acid sequences specifying the different chains of the multi-chain protein. This prevents the incorrect conception by the trained machine learning model that the concatenated amino acid sequence represents only a single chain, as opposed to multiple chains, thereby enabling a more accurate prediction of the one or more properties of the multi-chain protein, as compared to the conventional techniques.

In some embodiments, the techniques developed by the inventors include techniques for training a machine learning model to predict one or more properties of a multi-chain protein. In some embodiments, the techniques for training the machine learning model include generating training data and training the machine learning model using the generated training data. In some embodiments, generating the training data includes: (a) obtaining initial data for a plurality of multi-chain proteins that indicates, for each multi-chain protein, one or more properties of the particular multi-chain protein and sequence data that indicates a respective amino acid sequence for each chain of the particular multi-chain protein, (b) augmenting the initial data to obtain augmented data, and (c) encoding the augmented data to obtain the training data. The augmenting includes, for each multi-chain protein, (i) generating a concatenated amino acid sequence for the particular multi-chain protein at least in part by concatenating a linker amino acid sequence and the respective amino acid sequences indicated for each chain of the multi-chain protein, and/or (ii) generating permutations of the respective amino acid sequences indicated for each chain of the multi-chain protein.

In some embodiments, augmenting the initial data means, at a basic level, generating other amino acid sequences for a multi-chain protein that, while different from the initial amino acid sequence, would not be expected to change (materially or at all) the value of the property for the resulting multi-chain protein. Augmenting the initial data provides for multiple improvements over the conventional computational techniques for predicting properties of multi-chain proteins. For example, augmenting the initial data increases the amount of training data available to train the one or more machine learning models without requiring that additional proteins be expressed, produced, or manufactured, and their properties measured. This both reduces the expense of generating training data and makes training feasible. As another example, when the augmented data includes multiple permutations for a multi-chain protein, and the machine learning model is trained on representations of the multiple permutations, the trained machine learning model is capable of accurately predicting the one or more properties of the multi-chain protein regardless of the manner in which the multi-chain protein is encoded and presented to the trained machine learning model.

Following below are descriptions of various concepts related to, and embodiments of, techniques for predicting one or more properties of a multi-chain protein. It should be appreciated that various aspects described herein may be implemented in any of numerous ways, as the techniques are not limited in any particular manner of implementation. Example details of implementations are provided herein solely for illustrative purposes. Furthermore, the techniques disclosed herein may be used individually or in any suitable combination, as aspects of the technology described herein are not limited to the use of any particular technique or combination of techniques.

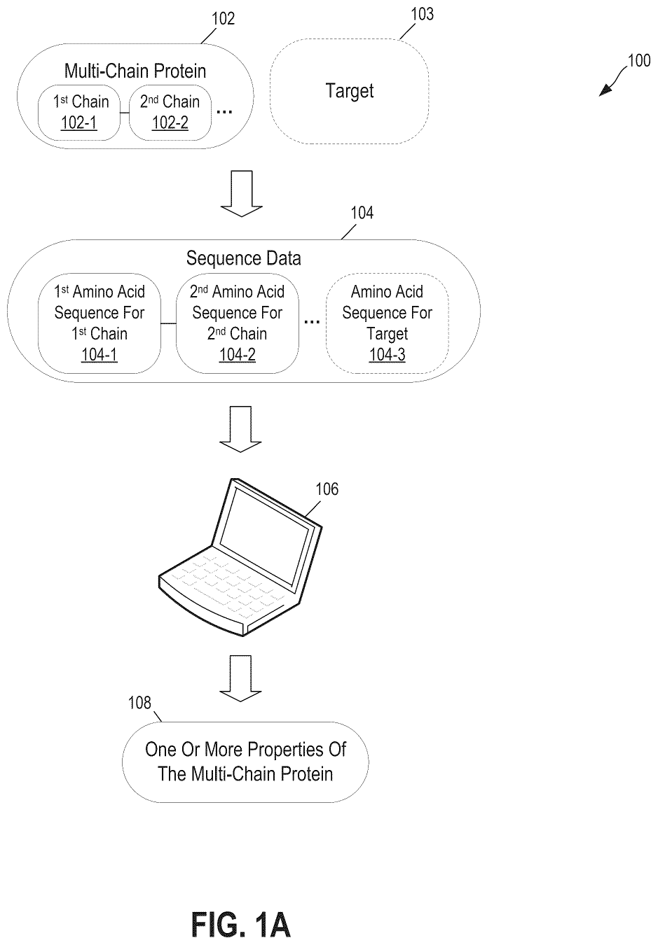

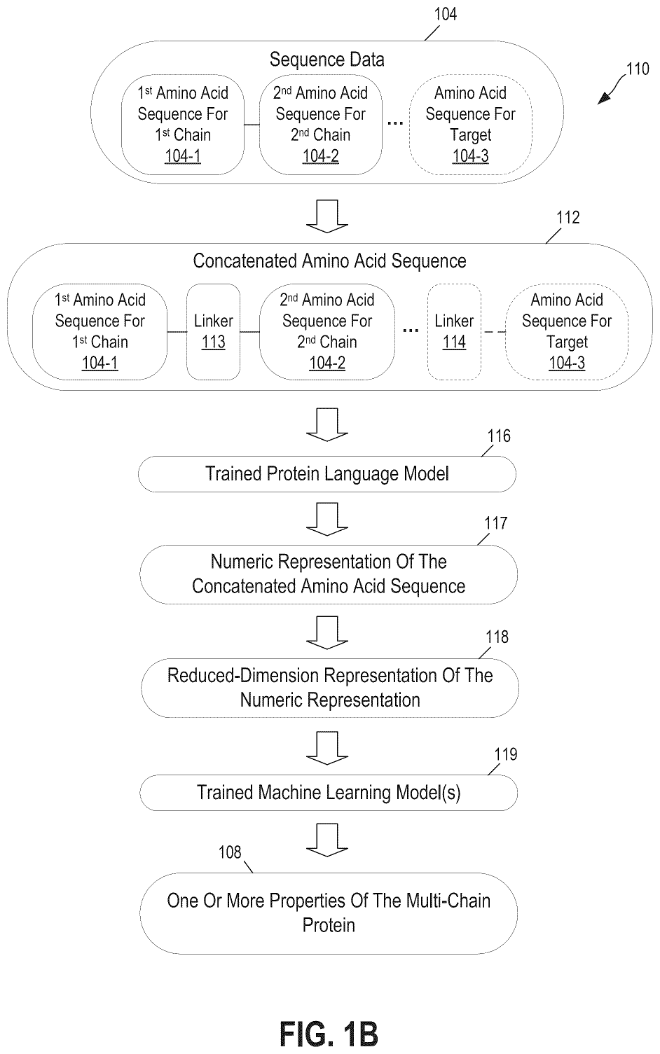

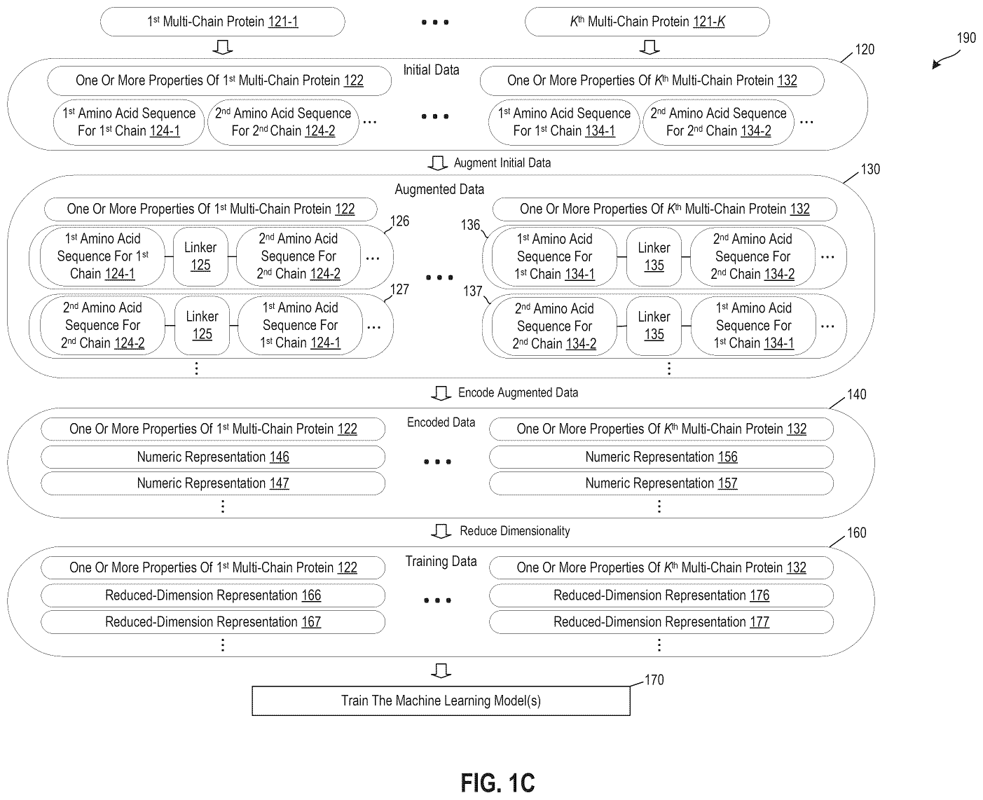

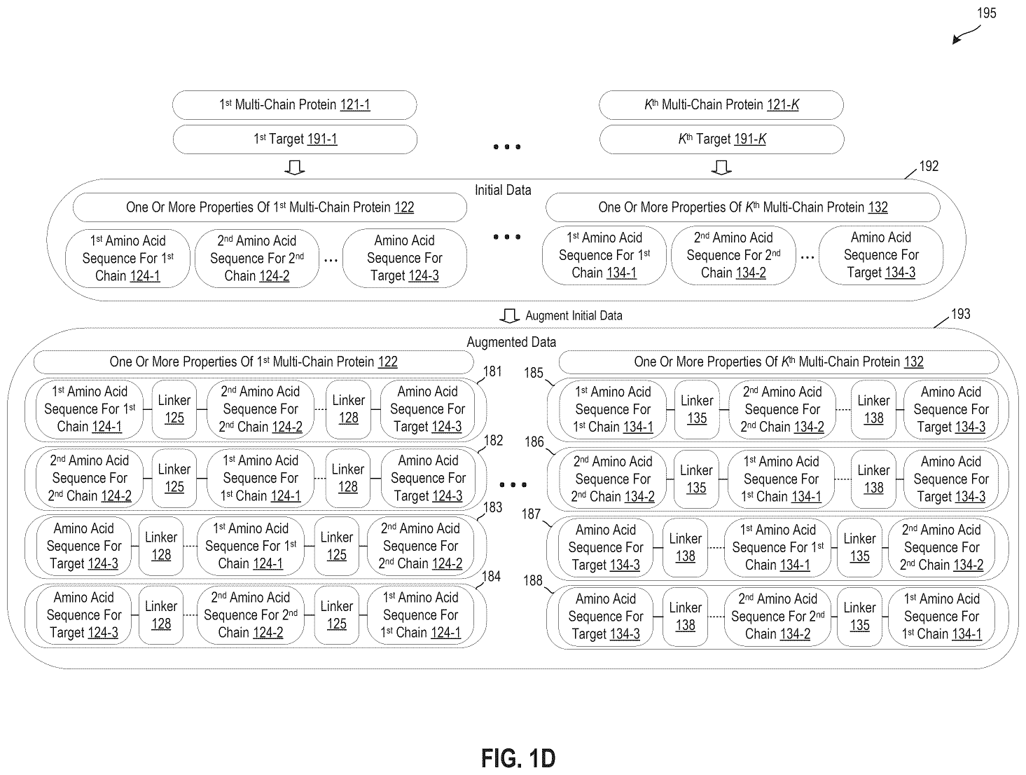

FIG. 1A is a diagram depicting an illustrative technique 100 for predicting one or more properties of a multi-chain protein, according to some embodiments of the technology described herein. Illustrative technique 100 includes obtaining sequence data 104 for a multi-chain protein and (optionally) target 103 (e.g., antigen, receptor, or other proteins) and processing the sequence data 104 using computing device 106 to predict one or more properties 108 of the multi-chain protein 102. In some embodiments, the one or more properties 108 may inform one or more decisions with respect to multi-chain protein 102 including, for example, whether to express, produce, or manufacture the multi-chain protein 102, further study the protein in one or more laboratory experiments, and/or whether to continue to invest resources, including human and capital resources, in investigating the utility of multi-chain protein 102 in one or more applications.

In some embodiments, illustrative technique 100 may be implemented in a laboratory setting. For example, aspects of the illustrative technique 100 may be implemented on a computing device 106 that is located within the laboratory setting. In some embodiments, the computing device 106 may directly obtain the sequence data 104 from a user (e.g., by the user uploading the sequence data 104 and/or interacting with computing device 106) within the laboratory setting. In some embodiments, the computing device 106 may indirectly obtain the sequence data 104 from another device (e.g., another computing device) within the laboratory setting. For example, the computing device 106 may obtain the sequence data 104 via at least one communication network, such as the Internet or any other suitable communication network(s), as aspects of the technology described herein are not limited in this respect.

In some embodiments, aspects of the illustrative technique 100 may be implemented in a setting that is located external to a laboratory setting. In this case, the computing device 106 may indirectly obtain the sequence data 104 from a computing device located within or externally to a laboratory setting. For example, the sequence data 104 may be provided to the computing device 106 via at least one communication network such as the Internet or any other suitable communication network(s), as aspects of the technology described herein are not limited in this respect.

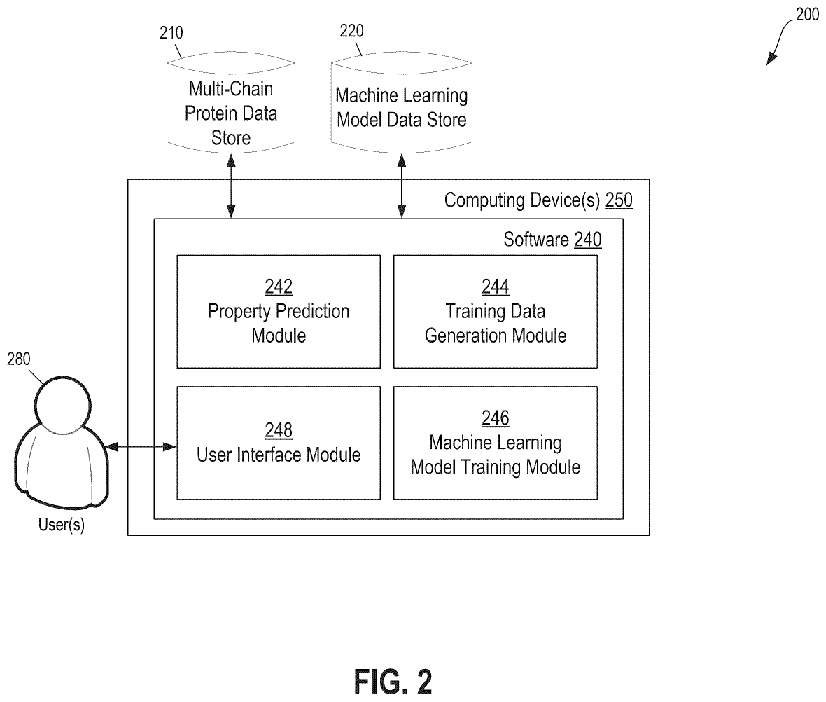

As shown in FIG. 1A, illustrative technique 100 includes obtaining sequence data 104 for a multi-chain protein 102. The multi-chain protein 102 has at least two chains (e.g., 2 chains, 3 chains, 4 chains, etc.). For example, as shown in FIG. 2, multi-chain protein 102 includes a first chain 102-1 and a second chain 102-2. However, it should be appreciated that the multi-chain protein 102 may include any number of chains suitable for a multi-chain protein, as aspects of the technology described herein are not limited in this respect. In some embodiments, the multi-chain protein 102 is a candidate multi-chain protein, meaning that it or a fragment of the multi-chain protein has not yet been expressed, produced, or manufactured (e.g., in vitro or in vivo). For example, the multi-chain protein 102 may be one of multiple candidate multi-chain proteins that have not yet been expressed, produced or manufactured. As described herein, the one or more predicted properties 108 of such a candidate multi-chain protein may be used to determine whether or not to proceed with expressing, producing, or manufacturing the multi-chain protein. In some embodiments, the one or more predicted properties 108 are determined for a library of candidate multi-chain proteins, and a candidate multi-chain protein is selected from the library for testing (e.g., performing an in vitro assay) to confirm if the candidate multi-chain protein has the one or more predicted properties 108. Aspects and examples of multi-chain proteins are described herein including at least in the section “Multi-Chain Proteins.” In some embodiments, illustrative technique 100 (optionally) includes obtaining sequence data 104 for a target 103.

In some embodiments, the sequence data 104 indicates an amino acid sequence for at least a portion (e.g., some or all) of each chain of the multi-chain protein 102 and (optionally) at least a portion (e.g., some or all) of a target 103. For example, as shown in FIG. 1A, sequence data 104 indicates a first amino acid sequence 104-1 for at least a portion of the first chain 102-1 (e.g., only a portion of first chain 102-1 or the entirety of the first chain 102-1) and a second amino acid sequence 104-2 for at least a portion of the second chain 102-2 (e.g., only a portion of the second chain 102-2 or the entirety of the second chain 102-2). Additionally, sequence data 104 indicates an amino acid sequence 104-3 for target 103.

In some embodiments, the sequence data 104 indicates a nucleic acid sequence for at least a portion (e.g., some or all) of each chain of the multi-chain protein 102 and (optionally) at least a portion (e.g., some or all) of the target 103. For example, the nucleic acid sequence for a portion of a chain may include a nucleic acid sequence that corresponds to an amino acid sequence for the portion of the chain. In some embodiments, a nucleic acid sequence that corresponds to a particular amino acid sequence includes a nucleic acid sequence that encodes the particular amino acid sequence (e.g., the nucleic acid sequence can be transcribed into mRNA, which can then be translated into the particular amino acid sequence). While embodiments of the technology described herein refer to using amino acid sequences (e.g., processing amino acid sequences using one or more machine learning models), it should be appreciated that nucleic acid sequence(s) may be used instead of amino acid sequence(s).

As described above, and as shown in FIG. 1A, illustrative technique 100 includes obtaining sequence data 104 for multi-chain protein 102 and (optionally) target 103. In some embodiments, a user (e.g., a researcher) and/or software may identify the sequence data 104 from among sequence data for multiple multi-chain proteins and/or targets. Additionally, or alternatively, the user and/or software may generate the sequence data by modifying amino acid sequence(s) (or nucleic acid sequence(s)) of multi-chain protein(s) having one or more known properties. Additionally, or alternatively, a trained generative machine learning model (e.g., a protein language model such as, for example, ProGen, ProGen2, and ProtGPT2) may be used to generate the sequence data 104. ProGen is described by Madani, A., et al. (“Progen: Language modeling for protein generation.” arXiv preprint arXiv: 2004.03497 (2020)), which is incorporated by reference herein in its entirety. ProGen2 is described by Nijkamp, E., et al. (“ProGen2: exploring the boundaries of protein language models.” Cell Systems 14.11 (2023): 968-978), which is incorporated by reference herein in its entirety. ProtGPT2 is described by Ferruz, N., et al. (“ProtGPT2 is a deep unsupervised language model for protein design.” Nature communications 13.1 (2022): 4348), which is incorporated by reference herein in its entirety. As yet another example, the sequence data 104 may be obtained by sequencing a multi-chain protein and/or target using a protein sequencing platform. In some embodiments, the sequence data 104 is otherwise obtained using any other suitable techniques, as aspects of the technology described herein is not limited in this respect.

As shown in the example of FIG. 1A, a computing device 106 may be used to process the sequence data 104 to obtain the one or more properties 108 of the multi-chain protein 102. In some embodiments, the computing device 106 may be operated by a user. For example, the user may provide sequence data 104 as input to the computing device 106 (e.g., by uploading a file) and/or provide user input specifying processing or other methods to be performed on sequence data 104. In other embodiments, computing device 106 may perform one or more calculations with respect to the sequence data without user intervention and, for example, can do so in response to receiving a request from a software program (e.g., via an API call) to do so.

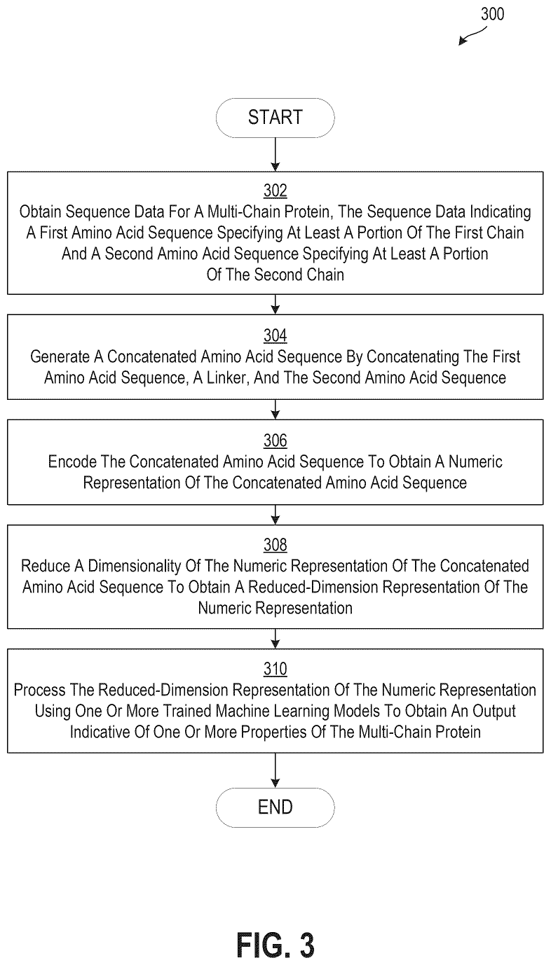

In some embodiments, software on the computing device 106 may be configured to process at least some (e.g., all) of the sequence data 104 to predict at least one of the one or more properties 108 of multi-chain protein 102. In some embodiments, this may include: (a) generating a concatenated amino acid sequence by concatenating one or more linkers (e.g., amino acid sequence, one or more tokens), the amino acid sequences indicated for the chains of multi-chain protein 102, and (optionally) target 103; (b) encoding the concatenated amino acid sequence to obtain a numeric representation of the concatenated amino acid sequence; and (c) processing the numeric representation of the concatenated amino acid sequence using a trained machine learning model to obtain an output indicative of the one or more properties of the multi-chain protein. Additionally, or alternatively, in some embodiments, processing the sequence data 104 to predict at least one of the one or more properties 108 of the multi-chain protein 102 includes: (a) generating a concatenated nucleic acid sequence by concatenating one or more linkers, the nucleic acid sequences indicated for the chains of multi-chain protein 102, and (optionally) target 103; (b) encoding the concatenated nucleic acid sequences to obtain a numeric representation of the concatenated nucleic acid sequence; and (c) processing the numeric representation of the concatenated nucleic acid sequence using a trained machine learning model to obtain an output indicative of the one or more properties of the multi-chain protein. Example techniques for predicting one or more properties of a multi-chain protein are described herein including at least with respect to FIG. 1B and FIG. 3.

In some embodiments, software on computing device 106 may be further configured to train one or more machine learning models to predict one or more properties of a multi-chain protein. It should be appreciated that a machine learning model can be used to predict a single property or multiple properties of a multi-chain protein, as aspects of the technology described herein are not limited to training machine learning models that predict only a single property for any particular multi-chain protein.

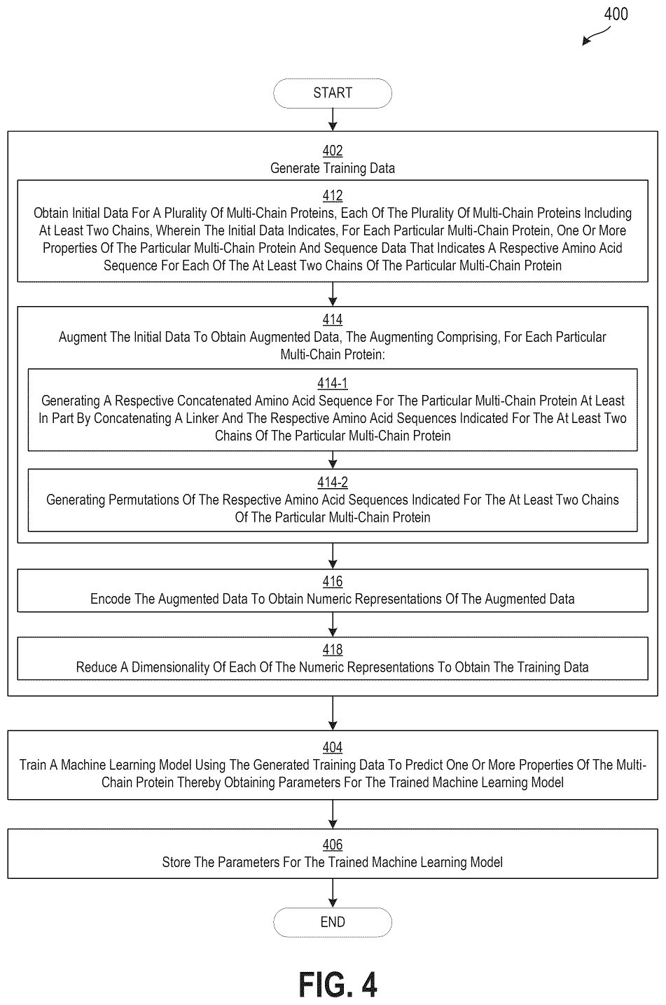

In some embodiments, training the one or more machine learning models to predict the one or more properties of the multi-chain protein includes: (a) generating training data; (b) training the machine learning model using the generated training data to predict one or more properties of the multi-chain protein thereby obtaining values for parameters of the trained machine learning model; and (c) storing the parameter values for the trained machine learning model. Generating the training data may include: (a) obtaining initial data indicating, for each of multiple multi-chain proteins (or optionally for each of multiple multi-chain protein and target pairs), one or more properties (e.g., one or more known properties) of the particular multi-chain protein and sequence data indicating a respective amino acid sequence for each chain of the particular multi-chain protein and (optionally) for each target; (b) augmenting the initial data to obtain augmented data; and (c) encoding the augmented data to obtain training data. The augmenting may include, for each particular multi-chain protein (or optionally for each particular multi-chain protein and target pair), generating permutations of the respective amino acid sequences indicated for the chains of the particular multi-chain protein and (optionally) the target and/or generating a respective concatenated amino acid sequence for the particular multi-chain protein and (optionally) the target using a linker. Additionally, or alternatively, generating the training data may include: (a) obtaining initial data indicating, for each of multiple multi-chain proteins (or optionally for each of multiple multi-chain protein and target pairs), one or more properties (e.g., one or more known properties) of the particular multi-chain protein and sequence data indicating a respective nucleic acid sequence for each chain of the particular multi-chain protein and (optionally) the target; (b) augmenting the initial data to obtain augmented data; and (c) encoding the augmented data to obtain training data. The augmenting may include, for each particular multi-chain protein (or optionally for each of multiple multi-chain protein and target pairs), generating permutations of the respective nucleic acid sequences indicated for the chains of the particular multi-chain protein and (optionally) the target and/or generating a respective concatenated nucleic acid sequence for the particular multi-chain protein and (optionally) the target using a linker. Example techniques for training a machine learning model to predict one or more properties of a multi-chain protein are described herein including at least with respect to FIG. 1C, FIG. 1D, and FIG. 4.

In some embodiments, software on computing device 106 may be configured to predict the one or more properties 108 of the multi-chain protein 102 from sequence data 104 and, optionally, one or more other inputs. Additionally, the software on computing device 106 may be configured to train one or more machine learning models to predict one or more properties of a multi-chain protein, and/or generate training data used to train such a machine learning model. An example of computing device 106 and such software is described herein including at least with respect to FIG. 2 (e.g., computing device(s) 250 and software 240).

As shown in FIG. 1A, the computing device 106 is configured to generate an output indicating one or more predicted properties 108 of the multi-chain protein 102. In some embodiments, the output may be stored (e.g., in memory), displayed via a user interface, transmitted to one or more other devices, used to generate a report, and/or otherwise processed using any other suitable techniques, as aspects of the technology described herein are not limited in this respect. For example, the output of the computing device 106 may be displayed using a graphical user interface (GUI) of a computing device (e.g., computing device 106).

In some embodiments, the computing device 106 includes one or multiple computing devices. In some embodiments, when the computing device 106 includes multiple computing devices, each of the computing devices may be used to perform the same process or processes. For example, each of the multiple computing devices may include software used to implement process 300 shown in FIG. 3 and/or process 400 shown in FIG. 4. In some embodiments, when the computing device 106 includes multiple computing devices, the computing devices may be used to perform different processes or different acts of a process. For example, one computing device may be used to implement process 300 shown in FIG. 3 and a different computing device may be used to implement process 400 shown in FIG. 4.

In some embodiments, when the computing device 106 includes multiple computing devices, the multiple computing devices may be configured to communicate via at least one communication network such as the Internet or any other suitable communication network(s), as aspects of the technology described herein are not limited in this respect. For example, the multiple computing devices may be part of a cloud computing environment. The cloud computing environment may be a public cloud computing environment, a private computing environment or a hybrid computing environment operating using a combination of publicly-accessible and private infrastructure.

Examples of the one or more properties 108 of the multi-chain protein 102 include viscosity, aggregation, stability, thermostability, three-dimensional structure, secondary structure, solubility, protein-protein interactions, disorder, accessible surface area, immunogenicity, pharmacokinetic clearance, bioavailability, productivity (e.g., yield), affinity, epitope-paratope mappings, chemical liability (e.g., oxidation) and endocytosis (e.g., specific, non-specific) and/or any other suitable property of multi-chain protein 102, as aspects of the technology described herein are not limited in this respect. Additionally or alternatively, the one or more properties 108 may be indicative of interactions between the multi-chain protein 102 and the target 103. For example, the one or more properties 108 may include binding affinity, specificity, binding site prediction, structural compatibility, and/or any other suitable properties indicative of the interactions between the multi-chain protein 102 and the target 103, as aspects of the technology described herein are not limited in this respect.

In some embodiments, the one or more predicted properties 108 may inform one or more decisions with respect to multi-chain protein 102 including, for example, whether to express, produce, or manufacture the multi-chain protein 102, further study the protein in one or more laboratory experiments, and/or whether to continue to invest resources, including human and capital resources, in investigating the utility of multi-chain protein 102 in one or more applications. For example, if the one or more predicted properties 108 satisfy specified criteria, the illustrative technique 100 may include taking an action (e.g., manufacturing, investing resources, further studying, investigating the utility of the multi-chain protein, etc.). Alternatively, if the one or more predicted properties 108 do not satisfy the specified criteria, the illustrative technique 100 may include repeating one or more acts of illustrative technique 100 to predict one or more properties of a different multi-chain protein.

In some embodiments, the one or more predicted properties 108 are used to identify one or more multi-chain proteins different from multi-chain protein 102. For example, one or more residues of the amino acid sequence(s) specifying chain(s) 102-1, 102-2 of multi-chain protein 102 may be modified to obtain amino acid sequence(s) specifying chain(s) of a different multi-chain protein. In some embodiments, the modifications are made based on the one or more predicted properties 108. For example, the residues may be modified to identify a multi-chain protein having improved properties relative to the properties 108 of multi-chain protein 102. In some embodiments, after modifying the residues or otherwise identifying a different multi-chain protein, one or more acts of illustrative technique 100 may be repeated for the identified multi-chain protein.

FIG. 1B is a diagram depicting an illustrative technique 110 for processing sequence data 104 to predict the one or more properties 108 of the multi-chain protein 102. The illustrative technique 110 includes: (a) concatenating the amino acid sequences indicated by sequence data 104 to obtain concatenated amino acid sequence 112, (b) encoding the concatenated amino acid sequence 112 to obtain a numeric representation 117 of the concatenated amino acid sequence 112, (c) reducing the dimensionality of the numeric representation 117 to obtain a reduced-dimension representation 118 of the numeric representation, and (d) processing the reduced-dimension representation 118 of the numeric representation using trained machine learning model(s) 119 to obtain the output indicative of the one or more properties 108 of the multi-chain protein. While illustrative technique 110 refers to using amino acid sequences to generate the data (e.g., reduced-dimension representation 118) that is processed using machine learning model(s) 119, it should be appreciated that nucleic acid sequences (e.g., indicated by sequence data 104) may instead be used to generate the data that is processed using machine learning model(s) 119. As described herein, including at least with respect to FIG. 1A, technique 110 may be implemented using a computing device such as computing device 106 shown in FIG. 1A.

As shown in FIG. 1B, the amino acid sequences indicated by sequence data 104 are concatenated to obtain concatenated amino acid sequence 112. In some embodiments, one or more linkers (e.g., amino acid sequence, one or more tokens) are concatenated between the amino acid sequences indicated by the sequence data. For example, in concatenated amino acid sequence 112, linker 113 is concatenated between the first amino acid sequence 104-1 and the second amino acid sequence 104-2. Additionally, when predicting properties of a multi-chain protein indicative of the interaction between the multi-chain protein 102 and the target 103, the concatenated amino acid sequence 112 may include a linker 114 concatenated between the amino acid sequences 104-1 and 104-2 corresponding to the multi-chain protein and the amino acid sequence 104-3 corresponding to the target 103. It should be appreciated, however, that concatenated amino acid sequence 112 may include one or more other linkers. For example, if sequence data 104 includes three amino acid sequences for three chains of the multi-chain protein, then a first linker may be concatenated between the first amino acid sequence and the second amino acid sequence, and a second linker may be concatenated between the second amino acid sequence and the third amino acid sequence.

As described herein, the linker 113 may be included in the concatenated amino acid sequence to distinguish between the first amino acid sequence 104-1 and the second amino acid sequence 104-2. This helps to ensure that the multi-chain protein is perceived as having multiple chains, rather than a single chain, which in turn improves the encoding and resulting numeric representation 117 of the multi-chain protein. Additionally or alternatively, linker 114 may be included in the concatenated amino acid sequence to distinguish between the multi-chain protein 102 and the target 103. Linker 113 and linker 114 may have different formats (e.g., different tokens, different lengths) to distinguish between chains in the multi-chain protein 102 and the target 103. In some embodiments, a linker (e.g., the linker 113 and linker 114) includes a sequence of one or more mask tokens such as one or more symbols and/or any other suitable type of token used to distinguish between different chains. For example, the one or more mask tokens may be one or more symbols including, for example, “|”, “@”, “\hat{ }”, “/”, “#”, “\”, or any other suitable symbol or sequence of symbols. Alternatively, the linker may include a sequence of one or more alanine amino acids (e.g., a poly-alanine linker). Alternatively, the linker may include any other suitable linker, as aspects of the technology described herein are not limited in this respect. The linker may be of any suitable length, as aspects of the technology described herein are not limited in this respect. For example, the linker may be at least 10, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, at least 200, or at least any other suitable number of amino acids or mask tokens, as aspects of the technology described herein are not limited in this respect. In some embodiments, the linker may be at most 200, at most 190, at most 180, at most 170, at most 160, at most 150, at most 140, at most 130, at most 120, at most 110, at most 100, at most 90, at most 80, at most 70, at most 60, at most 50, at most 40, at most 30, at most 20, at most 10, or at most any other suitable number of amino acids or mask tokens, as aspects of the technology described herein are not limited in this respect. It should be appreciated that any of the above-listed upper bounds may be coupled with any of the above-listed lower bounds. For example, the linker may have a length between 10 and 200 amino acids or mask tokens, between 50 and 150 amino acids or mask tokens, or between 80 and 100 amino acids or mask tokens.

As shown in FIG. 1B, concatenated amino acid sequence 112 is encoded to obtain the numeric representation 117 of the concatenated amino acid sequence 112. In some embodiments, the concatenated amino acid sequence 112 is encoded using a protein language model 116. The protein language model 116 may be any suitable protein language model trained to encode amino acid sequences by processing information representing an amino acid sequence to obtain a numeric output (e.g., a vector of real numbers) representing the encoding of the amino acid sequence, as aspects of the technology described herein are not limited in this respect. Examples of protein language models include AMPLIFY, the ESM-1b model, the ESM-1v model, the ESM-2 model, the ESM 3 model, and the ESM Cambrian model. AMPLIFY is described by Fournier, Q., et al. (“Protein language models: is scaling necessary?.” bioRxiv (2024): 2024-09), which is incorporated by reference herein in its entirety. The ESM-1b model is described by Rives, A., et al. (“Biological structure and function emerge from scaling unsupervised learning to 250 million protein sequences.” Proceedings of the National Academy of Sciences 118.15 (2021): e2016239118.), which is incorporated by reference herein in its entirety. The ESM-1v model is described by Meier, J., et al. (“Language models enable zero-shot prediction of the effects of mutations on protein function.” Advances in Neural Information Processing Systems 34 (2021): 29287-29303.), which is incorporated by reference herein in its entirety. The ESM-2 model is described by Lin, Z., et al. (“Evolutionary-scale prediction of atomic-level protein structure with a language model.” Science 379.6637 (2023): 1123-1130.), which is incorporated by reference herein in its entirety. ESM 3 is described by Hayes, Thomas, et al. (“Simulating 500 million years of evolution with a language model.” Science (2025): eads0018), which is incorporated by reference herein in its entirety. ESM Cambrian is described by ESM Team. (“ESM Cambrian: Revealing the mysteries of proteins with unsupervised learning.” EvolutionaryScale Website, Dec. 4, 2024. evolutionaryscale.ai/blog/esm-cambrian.), which is incorporated by reference herein in its entirety. In some embodiments, the concatenated amino acid sequence is encoded using one-hot encoding, label encoding, k-mer encoding, or any other suitable encoding techniques, as aspects of the technology described herein are not limited in this respect. In some embodiments, when nucleic acid sequences are used instead of amino acid sequences, one-hot encoding, label encoding, k-mer encoding, or another suitable encoding technique is used instead of a protein language model to encode the nucleic acid sequences.

In some embodiments, the dimensionality of the numeric representation 117 of the concatenated amino acid sequence 112 is reduced to obtain the reduced-dimension representation 118 of the numeric representation. The dimensionality may be reduced using any suitable dimensionality reduction technique(s), as aspects of the technology described herein are not limited to any particular dimensionality reduction techniques. Examples of dimensionality reduction technique(s) include principal components analysis (PCA), non-negative matrix factorization (NMF), kernel PCA, graph-based kernel PCA, multidimensional scaling (MDS), linear discriminant analysis (LDA), generalized discriminant analysis (GDA), and t-distributed stochastic neighbor embedding (t-SNE). When PCA is used, any suitable number of components may be selected, as aspects of the technology described herein are not limited in this respect. For example, the number of selected components may depend on the number of components capturing a specified percentage of variation in the data. In some embodiments, the number of selected components may include the number of components capturing at least 75%, at least 80%, at least 85%, at least 90%, at least 95%, at 97%, at least 98%, at least 99% or at least any other suitable percentage of variation in the data. In some embodiments, the number of selected components may include the number of components capturing at most 99%, at most 98%, at most 97%, at most 95%, at most 90%, at most 85%, at most 80%, at most 75%, or at most any other suitable percentage of variation in the data. It should be appreciated that any of the above-listed upper bounds may be coupled with any of the above-listed lower bounds. For example, the number of components may include a number of components capturing between 75% and 99% of the variation in the data, between 80% and 98% of the variation in the data, between 85% and 97% of the variation in the data, between 90% and 95% of the variation in the data, or a number of components capturing a percentage of variation in the data within any other suitable range.

As shown in FIG. 1B, the reduced-dimension representation 118 of the numeric representation 117 is processed using trained machine learning model(s) 119. The trained machine learning model(s) 119 are trained to predict the one or more properties 108 of the multi-chain protein (e.g., multi-chain protein shown in FIG. 1A). The trained machine learning model(s) 119 may include any type of machine learning model suitable for predicting one or more properties of a multi-chain protein, as aspects of the technology described herein are not limited in this respect. For example, the machine learning model may include any suitable type of classification or regression model. For example, the machine learning model may include a non-linear regression model (e.g., a logistic regression model), a linear regression model, a support vector machine, a Gaussian mixture model, a random forest model, a decision tree classifier, a gradient boosted decision tree classifier, a neural network model, and/or any other suitable type of machine learning model, as aspects of the technology described herein are not limited in this respect. Example machine learning models and techniques for training such models are described herein including at least in the section “Machine Learning” and with respect to FIG. 1C, FIG. 3, and FIG. 4.

It should be appreciated that illustrative technique 110 may include one or more additional or alternative acts. For example, illustrative technique 110 may include: (a) concatenating the first amino acid sequence 104-1, linker 113, second amino acid sequence 104-2, and (optionally) the linker 114 and amino acid sequence 104-3 to obtain the concatenated amino acid sequence 112; (b) encoding the concatenated amino acid sequence 112 to obtain the numeric representation 117 of the concatenated amino acid sequence 112; and (c) processing the numeric representation 117 using the trained machine learning model(s) 119 to obtain the output 108 indicative of the one or more properties of the multi-chain protein. Such embodiments may exclude the act of reducing the dimensionality of the numeric representation 117 to obtain the reduced-dimension representation 118 of the numeric representation 117. In some embodiments, illustrative technique 110 may include: (a) concatenating the first amino acid sequence 104-1 and the second amino acid sequence 104-2 to obtain the concatenated amino acid sequence 112; (b) encoding the concatenated amino acid sequence 112 to obtain the numeric representation 117 of the concatenated amino acid sequence 112; (c) reducing the dimensionality of the numeric representation 117 to obtain the reduced-dimension representation 118 of the numeric representation 117; and (d) processing the reduced-dimension representation 118 using the trained machine learning model(s) 119 to obtain the output 108 indicative of the one or more properties of the multi-chain protein. In such embodiments, the concatenated amino acid sequence 112 excludes the linker 113. In some embodiments, illustrative technique 110 may include: (a) concatenating the first amino acid sequence 104-1 and second amino acid sequence to obtain the concatenated amino acid sequence 112; (b) encoding the concatenated amino acid sequence 112 to obtain the numeric representation 117 of the concatenated amino acid sequence 112; and (c) processing the numeric representation 117 using the trained machine learning model(s) 119 to obtain the output 108 indicative of the one or more properties of the multi-chain protein. Such embodiments exclude both the linker 113 in the concatenated amino acid sequence 112 and the act of reducing the dimensionality of the numeric representation.

FIG. 1C is a diagram of an illustrative technique 190 for training a machine learning model to predict one or more properties of a multi-chain protein, according to some embodiments of the technology described herein. As shown in FIG. 1C, illustrative technique 190 includes: (a) obtaining initial data 120 for a plurality of multi-chain proteins (e.g., the first multi-chain protein 121-1 through the Kth multi-chain protein 121-K), (b) augmenting the initial data 120 to obtain augmented data 130, (c) encoding the augmented data to obtain encoded data 140, (d) reducing a dimensionality of the encoded data 140 to obtain the training data 160, and (e) training the machine learning model, at act 170, using the training data 160. While illustrative technique 190 refers to using amino acid sequences to generate the training data 160, it should be appreciated that nucleic acid sequences may instead be used to generate the training data 160.

As shown in FIG. 1C, the initial data 120 includes data for a plurality of multi-chain proteins including first multi-chain protein 121-1 through Kth multi-chain protein 121-K, where K is any number of multi-chain proteins suitable for training a machine learning model to predict one or more properties of a multi-chain protein, as aspects of the technology described herein are not limited to any particular number of multi-chain proteins. For example, K may be at least 10, at least 25, at least 50, at least 75, at least 90, at least 100, at least 125, at least 150, at least 175, at least 200, at least 225, at least 250, at least 260, at least 280, at least 290, at least 300, at least 310, at least 320, at least 330, at least 340, at least 350, at least 375, at least 400, at least 450, at least 500, at least 600, at least 700, at least 800, at least 900, at least 1,000, or at least any other suitable number of multi-chain proteins. In some embodiments, K may be at most 5,000, at most 2,500, at most 1,000, at most 900, at most 800, at most 800, at most 700, at most 600, at most 500, at most 450, at most 400, at most 375, at most 350, at most 340, at most 330, at most 320, at most 310, at most 300, at most 290, at most 280, at most 270, at most 260, at most 250, at most 200, at most 150, at most 100, at most 50, or at most any other suitable number of multi-chain proteins. It should be appreciated that any of the above-listed upper bounds may be coupled with any of the above-listed lower bounds. For example, K may be a number of multi-chain proteins between 50 and 7,500, between 90 and 5,000, between 150 and 2,500, between 200, and 1,000, between 250 and 500, between 280 and 300, or a number of multi-chain proteins within any other suitable range.

In some embodiments, the initial data 120 indicates, for each multi-chain protein: (a) one or more properties of the multi-chain protein and (b) sequence data for the multi-chain protein. In some embodiments, sequence data for a multi-chain protein includes a respective amino acid sequence for each chain of the multi-chain protein. Such an amino acid sequence may specify at least a portion (e.g., all) of a particular chain of the multi-chain protein. In some embodiments, the one or more properties of a particular multi-chain protein may include one or more properties measured in a lab, predicted using an accepted or verified method, or otherwise obtained using any other suitable techniques, as aspects of the technology described herein are not limited in this respect. The one or more properties may be the one or more properties which the machine learning model is trained to predict at act 170 and may include any suitable one or more properties, such as any of those described herein including at least with respect to FIG. 1A.

As shown in FIG. 1C, initial data 120 indicates, for the first multi-chain protein 121-1, one or more properties 122 of the first multi-chain protein and sequence data for the first multi-chain protein 121-1. The sequence data includes at least a first amino acid sequence 124-1 for the first chain of the of the first multi-chain protein 121-1 and a second amino acid sequence 124-2 for the second chain of the first multi-chain protein 121-1. It should be appreciated that the sequence data may indicate one or more additional amino acid sequences for one or more additional chains of the first multi-chain protein 121-1, as aspects of the technology described herein are not limited in this respect. The initial data 120 also indicates, for the Kth multi-chain protein 121-K, one or more properties 132 of the Kth multi-chain protein 121-K and sequence data for the Kth multi-chain protein 121-K. The sequence data includes a first amino acid sequence 134-1 for the first chain of the Kth multi-chain protein 121-K and a second amino acid sequence 134-2 for the second chain of the Kth multi-chain protein. It should be appreciated that the sequence data may indicate one or more additional amino acid sequences for one or more additional chains of the Kth multi-chain protein 121-K, as aspects of the technology described herein are not limited in this respect.

As shown in FIG. 1C, the initial data 120 is augmented to obtain augmented data 130. As described herein, augmenting the initial data means, at a basic level, generating other amino acid sequences for a multi-chain protein that, while different from the initial amino acid sequence, would not be expected to change (materially or at all) the value of the property for the resulting multi-chain protein. Augmenting the initial data has multiple benefits. For example, augmenting the initial data helps to increase the amount of training data available to train the one or more machine learning models without requiring that additional proteins be expressed, produced, or manufactured, and their properties measured. As another example, training the machine learning model using the augmented data enables the trained machine learning model to accurately and consistently predict one or more properties of a multi-chain protein regardless of the order in which the amino acid sequences (e.g., specifying the chains of the multi-chain protein) are encoded.

In some embodiments, augmenting the initial data 120 includes, for a multi-chain protein, concatenating one or more linkers (e.g., amino acid sequence, one or more tokens) and the amino acid sequences indicated for the chains of the multi-chain protein. For example, as shown in FIG. 1C, the first amino acid sequence 124-1, the linker 125, and the second amino acid sequence 124-2 are concatenated. The linker 125 may be concatenated between the first amino acid sequence 124-1 and the second amino acid sequence 124-2. Additionally, the first amino acid sequence 134-1, the linker 135, and the second amino acid sequence 134-2 are concatenated. The linker 135 may be concatenated between the first amino acid sequence 134-1 and the second amino acid sequence 134-2. While only a single linker is shown for each multi-chain protein (e.g., linker 125 for the first multi-chain protein 121-1 and linker 135 for the Kth multi-chain protein 121-K), it should be appreciated that more than one linker may be included. For example, where a multi-chain protein has three chains, the concatenated sequence may include two linkers (e.g., a first linker concatenated between a first amino acid sequence and a second amino acid sequence indicated for the first and second chains and a second linker concatenated between the second amino acid sequence and a third amino acid sequence indicated for the third chain).

Additionally, or alternatively, augmenting the initial data 120 may include, for each multi-chain protein, generating at least some (e.g., all) permutations of the amino acid sequences indicated for the chains of the particular multi-chain protein. Generating permutations of the amino acid sequences may include concatenating the amino acid sequences in different orders. For example, augmented data 130 shows permutations of each multi-chain protein. In particular, augmented data 130 shows a first permutation (e.g., concatenated sequence 126) and a second permutation (e.g., concatenated sequence 127) for the first multi-chain protein 121-1. The first permutation includes the first amino acid sequence 124-1, followed by linker 125, followed by the second amino acid sequence 124-2. The second permutation includes the second amino acid sequence 124-2 followed by linker 125, followed by the first amino acid sequence 124-1. Augmented data 130 also shows a first permutation (e.g., concatenated sequence 136) and a second permutation (e.g., concatenated sequence 137) for the Kth multi-chain protein 121-K. The first permutation includes the first amino acid sequence 134-1, followed by linker 135, followed by the second amino acid sequence 134-2. The second permutation includes the second amino acid sequence 134-2, followed by linker 135, followed by the first amino acid sequence 134-1. It should be appreciated that, while the augmented data 130 shows only two permutations for each multi-chain protein, the augmented data 130 may include more than two permutations. For example, the augmented data 130 may include more than two permutations for a multi-chain protein that includes more than two chains (e.g., up to six permutations for a multi-chain protein that includes three chains).

As shown in FIG. 1C, illustrative technique 190 further includes encoding the augmented data 130 to obtain encoded data 140. In some embodiments, encoding the augmented data 130 includes encoding the concatenated sequences generated for each of the multi-chain proteins. For example, encoding the augmented data 130 may include encoding concatenated sequence 126 to obtain the numeric representation 146 of the concatenated sequence 126, encoding the concatenated sequence 127 to obtain the numeric representation 147 of the concatenated sequence 127, encoding the concatenated sequence 136 to obtain the numeric representation 156 of concatenated sequence 136, and encoding the concatenated sequence 137 to obtain the numeric representation 157 of concatenated sequence 137. Example techniques for encoding a concatenated sequence are described herein including at least with respect to FIG. 1B, FIG. 3, and FIG. 4.

After encoding the augmented data 130, illustrative technique 190 includes reducing a dimensionality of the encoded data to obtain training data 160. In particular, this may include reducing the dimensionality of the numeric representations included in encoded data 140. For example, as shown in FIG. 1C, illustrative technique 190 may include reducing the dimensionality of numeric representation 146 to obtain the reduced-dimension representation 166, reducing the dimensionality of numeric representation 147 to obtain the reduced-dimension representation 167, reducing the dimensionality of numeric representation 156 to obtain the reduced-dimension representation 176, and reducing the dimensionality of numeric representation 157 to obtain the reduced-dimension representation 177. Example techniques for reducing the dimensionality of a numeric representation of a concatenated sequence are described herein including at least with respect to FIG. 1B, FIG. 3, and FIG. 4.

Training data 160 is used to train one or more machine learning models, at act 170, to predict one or more properties of multi-chain proteins. As described herein including at least with respect to FIG. 1B, the machine learning model may include any suitable machine learning model for predicting one or more properties of a multi-chain protein, as aspects of the technology described herein are not limited in this respect. In some embodiments, the machine learning model may be trained using any suitable training technique(s), including supervised techniques, semi-supervised techniques, unsupervised techniques, or any suitable combination thereof as aspects of the technology described herein are not limited in this respect. As one example, in the supervised training context, the reduced-dimension representation of a concatenated sequence generated for a multi-chain protein may be provided as input to the machine learning model, which may output one or more predicted properties of the multi-chain protein. Differences between the one or more predicted properties and the one or more known properties of the multi-chain protein (e.g., the one or more properties included in training data 160) may be used to determine and update the parameter values of the machine learning model. As one example, with reference to FIG. 1C, the reduced-dimension representation 166 may be provided as input to the machine learning model, which predicts one or more properties for the first multi-chain protein 121-1. The one or more predicted properties may be compared to the one or more properties 122 of the first multi-chain protein 121-1, and differences between the one or more predicted properties and the one or more properties 122 may be used to determine and/or update parameter values of the machine learning model.

It should be appreciated that illustrative technique 190 may include one or more additional or alternative acts. For example, illustrative technique 190 may exclude the act of reducing the dimensionality of the encoded data. In such an embodiment, the encoded data 140 may be used to train the one or more machine learning models at act 170. Additionally, or alternatively, augmenting the initial data may exclude the act of concatenating the linker with the amino acid sequences indicated for the chains of the multi-chain proteins. In such an embodiment, the amino acid sequences indicated for the chains of the multi-chain proteins may be concatenated without the linker.

As described herein, in some embodiments, a machine learning model is trained to predict one or more properties of a multi-chain protein that are indicative of the multi-chain protein's interaction with a particular target. In some embodiments, the inputs to such models include (i) amino acid sequences specifying chains of the multi-chain protein, and (ii) an amino acid sequence specifying the target. Training such a machine learning model may include steps for obtaining and augmenting initial data that are different from those shown in FIG. 1C. FIG. 1D is a diagram of an illustrative technique 195 for obtaining and augmenting data used to train a machine learning model to predict one or more properties of a multi-chain protein that are indicative of the multi-chain protein's interaction with a particular target.

As shown in FIG. 1D, illustrative technique 195 includes: (a) obtaining initial data 192 for a plurality of multi-chain protein and target pairs (e.g., the first multi-chain protein 121-1 and first target 191-1 through the Kth multi-chain protein 121-K and target 191-K), and (b) augmenting the initial data 192 to obtain augmented data 193. While illustrative technique 195 refers to using amino acid sequences to generate the training data, it should be appreciated that nucleic acid sequences may instead be used to generate the training data.

As shown in FIG. 1D, the initial data 192 includes data for a plurality of multi-chain protein and target pairs including a first pair (e.g., first multi-chain protein 121-1 and first target 191-1) through a Kth pair (e.g., Kth multi-chain protein 121-K and Kth target 191-K), where K is any number of pairs suitable for training a machine learning model to predict one or more properties of a multi-chain protein, as aspects of the technology described herein are not limited to any particular number of multi-chain proteins. For example, K may be at least 10, at least 25, at least 50, at least 75, at least 90, at least 100, at least 125, at least 150, at least 175, at least 200, at least 225, at least 250, at least 260, at least 280, at least 290, at least 300, at least 310, at least 320, at least 330, at least 340, at least 350, at least 375, at least 400, at least 450, at least 500, at least 600, at least 700, at least 800, at least 900, at least 1,000, or at least any other suitable number of pairs. In some embodiments, K may be at most 5,000, at most 2,500, at most 1,000, at most 900, at most 800, at most 800, at most 700, at most 600, at most 500, at most 450, at most 400, at most 375, at most 350, at most 340, at most 330, at most 320, at most 310, at most 300, at most 290, at most 280, at most 270, at most 260, at most 250, at most 200, at most 150, at most 100, at most 50, or at most any other suitable number of pairs. It should be appreciated that any of the above-listed upper bounds may be coupled with any of the above-listed lower bounds. For example, K may be a number of pairs between 50 and 7,500, between 90 and 5,000, between 150 and 2,500, between 200, and 1,000, between 250 and 500, between 280 and 300, or a number of pairs within any other suitable range.

In some embodiments, the initial data 192 indicates, for each multi-chain protein and target pair: (a) one or more properties of the multi-chain protein and (b) sequence data for the multi-chain protein and target. In some embodiments, sequence data for a multi-chain protein includes a respective amino acid sequence for each chain of the multi-chain protein. Such an amino acid sequence may specify at least a portion (e.g., all) of a particular chain of the multi-chain protein. In some embodiments, sequence data for a target includes an amino acid sequence for the target. The one or more properties 122 and 132 are described with respect to FIG. 1C.

As shown in FIG. 1D, initial data 192 indicates, for the first pair (e.g., first multi-chain protein 121-1 and first target 191-1), one or more properties 122 of the first multi-chain protein and sequence data for the first multi-chain protein 121-1 and the first target 191-1. The sequence data includes at least a first amino acid sequence 124-1 for the first chain of the first multi-chain protein 121-1, a second amino acid sequence 124-2 for the second chain of the first multi-chain protein 121-1, and a third amino acid sequence 124-3 for the target. It should be appreciated that the sequence data may indicate one or more additional amino acid sequences for one or more additional chains of the first multi-chain protein 121-1, as aspects of the technology described herein are not limited in this respect. The initial data 192 also indicates, for the Kth pair (e.g., the Kth multi-chain protein 121-K and the Kth target 191-K, one or more properties 132 of the Kth multi-chain protein 121-K and sequence data for the Kth multi-chain protein 121-K and the Kth target 191-K. The sequence data includes a first amino acid sequence 134-1 for the first chain of the Kth multi-chain protein 121-K, a second amino acid sequence 134-2 for the second chain of the Kth multi-chain protein, and a third amino acid sequence 134-3 for the target. It should be appreciated that the sequence data may indicate one or more additional amino acid sequences for one or more additional chains of the Kth multi-chain protein 121-K, as aspects of the technology described herein are not limited in this respect.

As shown in FIG. 1D, the initial data 192 is augmented to obtain augmented data 193. In some embodiments, augmenting the initial data 192 includes, for a particular multi-chain protein and target pair, concatenating one or more linkers (e.g., amino acid sequence, one or more tokens) and the amino acid sequences indicated for the chains of the multi-chain protein and the target. For example, as shown in FIG. 1D, the linker 125 is concatenated between the first amino acid sequence 124-1 and the second amino acid sequence 124-2. The linker 128 is concatenated between the set of amino acid sequences 124-1, 124-2 for the first multi-chain protein 121-1 and the amino acid sequence 124-3 for the target 191-1. The linker 128 is used to distinguish between the first multi-chain protein 121-1 and the first target 191-1. Additionally, the linker 135 is concatenated between the first amino acid sequence 134-1 and the second amino acid sequence 134-2. The linker 138 is concatenated between the set of amino acid sequences 134-1, 134-2 for the Kth multi-chain protein 121-K and the amino acid sequence 124-3 for the Kth target 191-K. The linker 138 is used to distinguish between the Kth multi-chain protein 121-K and the Kth target 191-K. In some embodiments, the same type of linker is used between chains of a multi-chain protein and between the multi-chain protein and the target for all pairs of multi-chain proteins and targets. For example, linkers 125, 128, 135, and 138 may be the same type of linker (e.g., same length, same token). In some embodiments, different types of linkers are used between chains of a multi-chain protein and between the multi-chain protein and the target. For example, linkers 125 and 135 may be a first linker type and linkers 128 and 138 may be a second linker type, where the first and second linker types are different (e.g., different lengths, different tokens).

Additionally, or alternatively, augmenting the initial data 192 may include, for each multi-chain protein, generating at least some permutations of the amino acid sequences indicated for the chains of the particular multi-chain protein and target. Generating permutations of the amino acid sequences may include concatenating the amino acid sequences in different orders. For example, augmented data 193 shows permutations of each multi-chain protein and target pair. In particular, augmented data 193 shows four permutations (e.g., concatenated sequences 181, 182, 183, 184) for the first multi-chain protein and target pair. The first permutation includes the first amino acid sequence 124-1, followed by linker 125, followed by the second amino acid sequence 124-2, followed by linker 128, followed by target amino acid sequence 124-3. The second permutation includes the second amino acid sequence 124-2, followed by linker 125, followed by the first amino acid sequence 124-1, followed by linker 128, followed by target amino acid sequence 124-3. The third permutation includes the target amino acid sequence 124-3, followed by linker 128, followed by the first amino acid sequence 124-1, followed by linker 125. The fourth permutation includes the target amino acid sequence 124-3, followed by linker 128, followed by the second amino acid sequence 124-2, followed by linker 125, followed by the first amino acid sequence 124-1. In this example, none of the permutations include the target amino acid sequence 124-3 concatenated between any pair of amino acid sequences specified for the first multi-chain protein. Rather, the target amino acid sequence 124-3 is concatenated via linker 128 to either end of the set of multi-chain protein sequences.

Augmented data 193 also shows four permutations (e.g., concatenated sequences 185, 186, 187, 188) for the Kth multi-chain protein and target pair. The Kth permutation includes the first amino acid sequence 134-1, followed by linker 135, followed by the second amino acid sequence 134-2, followed by linker 138, followed by target amino acid sequence 134-3. The second permutation includes the second amino acid sequence 134-2, followed by linker 135, followed by the first amino acid sequence 134-1, followed by linker 138, followed by target amino acid sequence 134-3. The third permutation includes the target amino acid sequence 134-3, followed by linker 138, followed by the first amino acid sequence 134-1, followed by linker 135, followed by the second amino acid sequence 134-2. The fourth permutation includes the target amino acid sequence 134-3, followed by linker 138, followed by the second amino acid sequence 134-2, followed by linker 135, followed by the first amino acid sequence 134-1. In this example, none of the permutations include the target amino acid sequence 134-3 concatenated between any pair of amino acid sequences specified for the Kth multi-chain protein. Rather, the target amino acid sequence 134-3 is concatenated via linker 128 to either end of the set of multi-chain protein sequences.

Table 1 provides an example of permutations of a target and multi-chain protein pair, where the multi-chain protein has two different chains specified by amino acid sequences “A” and “B”, the target is specified by amino acid sequence “C” and “L” denotes a linker:

| Permutation # | Concatenated Sequence |
|---|---|
| Permutation 1 | A-L-B-L-C |
| Permutation 2 | B-L-A-L-C |
| Permutation 3 | C-L-A-L-B |
| Permutation 4 | C-L-B-L-A |

In some embodiments, the augmented data 193 is used at acts 140, 160, and 170 to obtain a machine learning model trained to predict one or more properties of a multi-chain protein when providing target and multi-chain protein sequence data as input. FIG. 2 is a block diagram of an example system 200 for predicting one or more properties of a multi-chain protein, according to some embodiments of the technology described herein. System 200 includes computing device(s) 250 configured to have software 240 execute thereon to perform various functions in connection with training and using a machine learning model to predict one or more properties of a multi-chain protein. In some embodiments, the software 240 includes a plurality of modules. A module may include processor-executable instructions that, when executed by at least one computer hardware processor, cause the at least one computer hardware processor to perform function(s) of the module. Such modules are sometimes referred to herein as “software modules,” each of which includes processor-executable instructions configured to perform one or more acts of one or more processes, such as process 300 shown in FIG. 3 and process 400 shown in FIG. 4.

The computing device(s) 250 may be operated by one or more user(s) 280. In some embodiments, the user(s) 280 may provide, as input to the computing device(s) 250 (e.g., by uploading one or more files, by interacting with a user interface of the computing device(s) 250, etc.) data for one or more multi-chain proteins (e.g., sequence data, property data, etc.). Additionally, or alternatively, the user(s) 280 may provide input specifying processing or other methods to be performed on sequence data for a multi-chain protein. Additionally, or alternatively, the user(s) 280 may access results of processing the sequence data. For example, the user(s) 280 may access a prediction indicative of one or more properties of the multi-chain protein.

In some embodiments, the property prediction module 242 obtains sequence data for a multi-chain protein and (optionally) a target. For example, the property prediction module 242 may obtain the sequence data from the multi-chain protein data store 210, and/or user(s) 280. The sequence data may indicate amino acid sequences, each of which specifies at least a portion of a respective chain of the multi-chain protein and (optionally) the target. Additionally, or alternatively, the sequence data may indicate nucleic acid sequence(s) for chain(s) of the multi-chain protein and (optionally) the target.

In some embodiments, the property prediction module 242 is configured to predict one or more properties of the multi-chain protein using the sequence data obtained for the multi-chain protein and (optionally) the target. To this end, in some embodiments, the property prediction module 242 is configured to: (a) generate a concatenated sequence using the sequence data, (b) encode the concatenated sequence to obtain a numeric representation of the concatenated sequence, (c) reduce a dimensionality of the numeric representation to obtain a reduced-dimension representation of the numeric representation, and (d) process the reduced-dimension representation using a machine learning model trained to predict the one or more properties of the multi-chain protein. The concatenated sequence may refer to a concatenated amino acid sequence or a concatenated nucleic acid sequence.

As described herein, in some embodiments, the property prediction module 242 is configured to generate a concatenated sequence using the obtained sequence data. For example, the property prediction module 242 may be configured to generate a concatenated amino acid sequence by concatenating the amino acid sequences indicated by the obtained sequence data and one or more linkers (e.g., amino acid sequence, one or more tokens). Additionally, or alternatively, the property prediction module 242 may be configured to generate a concatenated nucleic acid sequence by concatenating the nucleic acid sequences indicated by the obtained sequence data and one or more linkers. Example techniques for generating a concatenated sequence are described herein including at least with respect to FIG. 1B and act 304 of process 300 shown in FIG. 3.

In some embodiments, the property prediction module 242 is further configured to encode the concatenated sequence to obtain a numeric representation of the concatenated sequence. In some embodiments, the property prediction module 242 is configured to obtain a protein language model (e.g., from machine learning model data store 220) and encode a concatenated amino acid sequence using the protein language model. For example, the property prediction module 242 may be configured to process the concatenated amino acid sequence using the AMPLIFY model, the ESM-1b model, the ESM-1v model, the ESM-2 model, the ESM 3 model, and/or the ESM Cambrian model to obtain the numeric representation of the concatenated amino acid sequence. In some embodiments, the property prediction module 242 is configured to encode the concatenated sequence using one-hot encoding, label encoding, k-mer encoding, or any other suitable encoding techniques, as aspects of the technology described herein are not limited in this respect. Example techniques for encoding a concatenated sequence are described herein including at least with respect to FIG. 1B and act 306 of process 300 shown in FIG. 3.

In some embodiments, the property prediction module 242 is further configured to reduce a dimensionality of the numeric representation of the concatenated sequence. The property prediction module 242 may be configured to implement any suitable dimensionality reduction technique(s), as aspects of the technology described herein are not limited in this respect. Example dimensionality reduction techniques are described herein including at least with respect to FIG. 1B.

In some embodiments, the property prediction module 242 is further configured to predict one or more properties of a multi-chain protein. For example, the property prediction module 242 may be configured to process the numeric representation (e.g., the reduced-dimension representation of the numeric representation) of the concatenated sequence generated for the multi-chain protein using a machine learning model trained to predict the one or more properties of the multi-chain protein. The machine learning model may include any suitable type of machine learning model, as aspects of the technology described herein are not limited in this respect. Examples of such machine learning models are described herein including at least with respect to FIG. 1B, act 310 of process 300 shown in FIG. 3, and in the section “Machine Learning.” In some embodiments, the property prediction module 242 is configured to obtain the trained machine learning model from the machine learning model data store 220 and/or the machine learning model training module 246.

In some embodiments, the training data generation module 244 is configured to generate training data used to train one or more machine learning models to predict one or more properties of a multi-chain protein. To this end, in some embodiments, the training data generation module 244 is configured to: (a) obtain initial data for a plurality of multi-chain proteins, (b) augment the initial data to obtain augmented data, (c) encode the augmented data to obtain encoded data, and (d) reduce a dimensionality of the encoded data.

In some embodiments, the training data generation module 244 is configured to obtain initial data for a plurality of multi-chain proteins. The training data generation module 244 may be configured to obtain the initial data from the user(s) 280 (e.g., by the user(s) 280 uploading the initial data) and/or the multi-chain protein data store 210. As described herein, in some embodiments, the initial data indicates, for each particular multi-chain protein (or multi-chain protein and target pair), one or more properties of the multi-chain protein and sequence data that indicates a respective amino acid sequence (or nucleic acid sequence) for each of the chains of the particular multi-chain protein and (optionally) target. Examples of the initial data are described herein including at least with respect to FIG. 1C, FIG. 1D, and act 412 of process 400 shown in FIG. 4.

In some embodiments, the training data generation module 244 is configured to augment the initial data. Augmenting the initial data may include, for each multi-chain protein (or multi-chain protein and target pair): (a) generating a respective concatenated sequence by concatenating one or more linkers and the respective sequences (e.g., amino acid sequences or nucleic acid sequences) indicated (e.g., in the initial data) for the chains of the multi-chain protein and (optionally) the target, and/or (b) generating permutations of the respective sequences indicated for the chains of the multi-chain protein and (optionally) the target. Examples of generating a concatenated sequence including one or more linkers are described herein including at least respect to FIG. 1C, FIG. 1D and act 414-1 of process 400 shown in FIG. 4. Examples of generating permutations of sequences are described herein including at least with respect to FIG. 1C, FIG. 1D, and act 414-2 of process 400 shown in FIG. 4.

In some embodiments, the training data generation module 244 is configured to encode the augmented data. In some embodiments, encoding the augmented data includes encoding the concatenated sequences generated for the multi-chain proteins to obtain numeric representations of the concatenated sequences. In some embodiments, the training data generation module 244 is configured to obtain a protein language model (e.g., from machine learning model data store 220) and encode concatenated amino acid sequences using the protein language model. For example, the training data generation module 244 may be configured to process the concatenated amino acid sequences using the AMPLIFY model, the ESM-1b model, the ESM-1v model, the ESM-2 model, the ESM 3 model, and/or the ESM Cambrian model to obtain the numeric representations of the concatenated amino acid sequences. In some embodiments, the training data generation module 244 is configured to encode the concatenated sequences using one-hot encoding, label encoding, k-mer encoding, or any other suitable encoding techniques, as aspects of the technology described herein are not limited in this respect. Example techniques for encoding a concatenated sequence are described herein including at least with respect to FIG. 1C and act 416 of process 400 shown in FIG. 4.

In some embodiments, the training data generation module 244 is configured to reduce a dimensionality of the encoded data. In some embodiments, reducing the dimensionality of the encoded data includes reducing the dimensionality of the numeric representations of the concatenated sequences. The training data generation module 244 may be configured to implement any suitable dimensionality reduction technique(s), as aspects of the technology described herein are not limited in this respect. Example dimensionality reduction techniques are described herein including at least with respect to FIG. 1B.

In some embodiments, the machine learning model training module 246 is configured to train one or more machine learning models to predict one or more properties of a multi-chain protein. For example, the machine learning model training module 246 may obtain training data from the training data generation module 244, multi-chain protein data store 210, and/or user(s) 280 (e.g., by the user(s) 280 uploading the training data). The machine learning model training module 246 may be configured to use the obtained training data to train the one or more machine learning models to predict one or more properties of a multi-chain protein. In some embodiments, the machine learning model training module 246 may provide the trained machine learning model(s) to the machine learning model data store 220 for storage thereon. For example, the machine learning model training module 246 may provide the values of the parameters of the machine learning model(s) to the machine learning model data store 220 for storage thereon. Example techniques for training a machine learning model to predict one or more properties of a multi-chain protein are described herein including at least with respect to FIG. 1C and FIG. 4.

As shown in FIG. 2, system 200 also includes multi-chain protein data store 210 and machine learning model data store 220. In some embodiments, software 240 obtains data from multi-chain protein data store 210, machine learning model data store 220, and/or user(s) 280 (e.g., by uploading one or more files).

In some embodiments, multi-chain protein data store 210 stores training data used to train one or more machine learning models to predict one or more properties of a multi-chain protein (e.g., training data generated by the training data generation module 244). Additionally, or alternatively, multi-chain protein data store 210 stores data about one or more candidate multi-chain proteins such as, for example, sequence data for the candidate multi-chain protein. Additionally, or alternatively, multi-chain protein data store 210 stores data about one or more targets such as, for example, sequence data for the targets. In some embodiments, the multi-chain protein data store 210 includes any suitable type of data store (e.g., a flat file, a database system, a multi-file, etc.) and may store data in any suitable format, as aspects of the technology described herein are not limited in this respect. The multi-chain protein data store 210 may be part of software 240 (not shown) or excluded from software 240, as shown in FIG. 2.

In some embodiments, the machine learning model data store 220 stores one or more machine learning models trained to predict a respective one or more properties of a multi-chain protein. Additionally, or alternatively, the machine learning model data store 220 stores one or more protein language models trained to encode amino acid sequences. In some embodiments, the machine learning model data store 220 includes any suitable type of data store such as a flat file, a database system, a multi-file, or data store of any suitable type, as aspects of the technology described herein are not limited to any particular type of data store. The machine learning model data store 220 may be part of software 240 (not shown) or excluded from software 240, as shown in FIG. 2. In some embodiments, the machine learning model data store 220 may store parameter values for trained machine learning model(s). When the stored trained machine learning model(s) are loaded and used, for example by property prediction module 242, the parameter values of the trained machine learning model are loaded and stored in memory using at least one data structure.

As shown in FIG. 2, software 240 also includes user interface module 248. User interface module 248 may be configured to generate a graphical user interface (GUI) through which user(s) 280 may provide input and view information generated by software 240. For example, in some embodiments, the user interface module 248 may be a webpage or web application accessible through an Internet browser. In some embodiments, the user interface module 248 may generate a GUI of an app executing on a user's mobile device. In some embodiments, the user interface module 248 may generate a number of selectable elements through which a user may interact. For example, the user interface module 248 may generate dropdown lists, checkboxes, text fields, or any other suitable element.

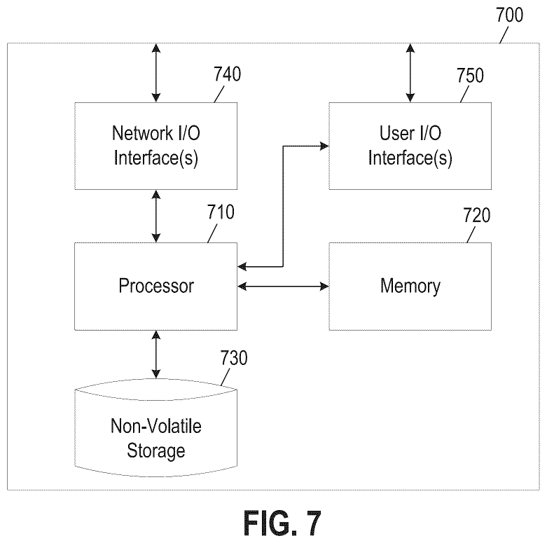

FIG. 3 is a flowchart of an illustrative process 300 for predicting one or more properties of a multi-chain protein, according to some embodiments of the technology described herein. One or more (e.g., all) of the acts of process 300 may be performed automatically by any suitable computing device(s). For example, the act(s) may be performed by a laptop computer, a desktop computer, one or more servers, in a cloud computing environment, computing device(s) 250 as described herein including at least with respect to FIG. 2, computing system 700 as described herein including at least with respect to FIG. 7, and/or in any other suitable way, as aspects of the technology described herein are not limited in this respect.

At act 302, sequence data is obtained for a multi-chain protein having any suitable number of chains. For example, the multi-chain protein may have at least two chains, at least three chains, at least four chains, at least five chains, at least six chains, at least seven chains, at least eight chains, at least nine chains, at least ten chains, or at least any other suitable number of chains. In some embodiments, the multi-chain protein may have at most ten chains, at most nine chains, at most eight chains, at most seven chains, at most six chains, at most five chains, at most four chains, at most three chains, at most two chains, or at most any other suitable number of chains. It should be appreciated that any of the above-listed upper bounds may be coupled with any of the above-listed lower bounds. For example, the multi-chain protein may have between two and twenty chains, between two and ten chains, between two and eight chains, between two and six chains, between two and four chains, or any number of chains within the above-listed ranges.

In some embodiments, the sequence data indicates, for each particular chain of the multi-chain protein, a respective amino acid sequence specifying at least a portion (e.g., all) of the particular chain. Consider, for example, a multi-chain protein having at least a first chain and a second chain. The sequence data may indicate a first amino acid sequence specifying at least a portion (e.g., all) of the first chain and a second amino acid sequence specifying at least a portion (e.g., all) of the second chain. In some embodiments, the sequence data indicates, for each particular chain of the multi-chain protein, a respective nucleic acid sequence corresponding to an amino acid sequence specifying at least a portion (e.g., all) of the particular chain. While embodiments of the technology described herein refer to using amino acid sequences (e.g., processing amino acid sequences using one or more machine learning models), it should be appreciated that nucleic acid sequence(s) may be used instead of amino acid sequence(s).

At act 304, a concatenated amino acid sequence is generated by concatenating at least one linker (e.g., amino acid sequence, one or more tokens) and the amino acid sequences specifying the portions of the chains of the multi-chain protein. For example, for a multi-chain protein including a first chain and a second chain, generating the concatenated amino acid sequence may include concatenating the first amino acid sequence specifying at least a portion of the first chain, a linker, and the second amino acid sequence specifying at least a portion of the second chain. In some embodiments, if the multi-chain protein includes more than two chains, then more than one linker may be included in the concatenated amino acid sequence. For example, for a multi-chain protein having a first chain, a second chain, and a third chain, generating the concatenated sequence may include: (a) concatenating a linker between the first amino acid sequence specifying at least a portion of the first chain and a second amino acid sequence specifying at least a portion of the second chain, and (b) concatenating a linker between the second amino acid sequence and a third amino acid sequence specifying at least a portion of the third chain. The linker may include any suitable linker of any suitable length, as aspects of the technology described herein are not limited in this respect. For example, the linker may include one or more mask tokens (e.g., one or more symbols, etc.) or one or more alanine amino acids (e.g., a poly-alanine). In some embodiments, the linker has a length of at least 10, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, at least 200, or at least any other suitable number of amino acids or mask tokens, as aspects of the technology described herein are not limited in this respect. In some embodiments, the linker has a length of at most 200, at most 190, at most 180, at most 170, at most 160, at most 150, at most 140, at most 130, at most 120, at most 110, at most 100, at most 90, at most 80, at most 70, at most 60, at most 50, at most 40, at most 30, at most 20, at most 10, or at most any other suitable number of amino acids or mask tokens, as aspects of the technology described herein are not limited in this respect. It should be appreciated that any of the above-listed upper bounds may be coupled with any of the above-listed lower bounds. For example, the linker may have a length between 10 and 200 amino acids or mask tokens, between 50 and 150 amino acids or mask tokens, between 80 and 100 amino acids or mask tokens, or a length within any other suitable range.

In some embodiments, act 304 includes generating the concatenated amino acid sequence without the linker. In such embodiments, generating the concatenated amino acid sequence includes concatenating the amino acid sequences specifying the portions of the chains of the multi-chain protein.

At act 306, the concatenated amino acid sequence is encoded to obtain a numeric representation of the concatenated amino acid sequence. In some embodiments, the concatenated amino acid sequence is encoded using a protein language model. The protein language model may be any suitable protein language model trained to encode amino acid sequences by processing information representing an amino acid sequence to obtain a numeric output (e.g., a vector of real numbers) representing the encoding of the amino acid sequence, as aspects of the technology described herein are not limited in this respect. Examples of protein language models include AMPLIFY, the ESM-1b model, the ESM-1v model, the ESM-2 model, the ESM 3 model, and the ESM Cambrian model. AMPLIFY is described by Fournier, Q., et al. (“Protein language models: is scaling necessary?.” bioRxiv (2024): 2024-09.), which is incorporated by reference herein in its entirety. The ESM-1b model is described by Rives, A., et al. (“Biological structure and function emerge from scaling unsupervised learning to 250 million protein sequences.” Proceedings of the National Academy of Sciences 118.15 (2021): e2016239118.), which is incorporated by reference herein in its entirety. The ESM-1v model is described by Meier, J., et al. (“Language models enable zero-shot prediction of the effects of mutations on protein function.” Advances in Neural Information Processing Systems 34 (2021): 29287-29303.), which is incorporated by reference herein in its entirety. The ESM-2 model is described by Lin, Z., et al. (“Evolutionary-scale prediction of atomic-level protein structure with a language model.” Science 379.6637 (2023): 1123-1130.), which is incorporated by reference herein in its entirety. ESM 3 is described by Hayes, Thomas, et al. (“Simulating 500 million years of evolution with a language model.” Science (2025): eads0018.), which is incorporated by reference herein in its entirety. ESM Cambrian is described by ESM Team. (“ESM Cambrian: Revealing the mysteries of proteins with unsupervised learning.” EvolutionaryScale Website, Dec. 4, 2024. evolutionaryscale.ai/blog/esm-cambrian.), which is incorporated by reference herein in its entirety. In some embodiments, the concatenated amino acid sequence is encoded using one-hot encoding, label encoding, k-mer encoding, or any other suitable encoding techniques, as aspects of the technology described herein are not limited in this respect. In some embodiments, when nucleic acid sequences are used instead of amino acid sequences, one-hot encoding, label encoding, k-mer encoding, or another suitable encoding technique is used instead of a protein language model.

At act 308, a dimensionality of the numeric representation of the concatenated amino acid sequence is reduced to obtain a reduced-dimension representation of the numeric representation. The dimensionality may be reduced using any suitable dimensionality reduction techniques, as aspects of the technology described herein are not limited to any particular dimensionality reduction techniques. Examples of dimensionality reduction technique(s) are described herein including at least with respect to FIG. 1B.

At act 310, the reduced-dimension representation of the numeric representation is processed using one or more trained machine learning models to obtain an output indicative of one or more properties of the multi-chain protein. The trained machine learning model(s) may include any type of machine learning model suitable for predicting one or more properties of a multi-chain protein, as aspects of the technology described herein are not limited in this respect. For example, the machine learning model(s) may include any suitable type of classification or regression model. For example, the machine learning model(s) may include a non-linear regression model (e.g., a logistic regression model), a linear regression model, a support vector machine, a Gaussian mixture model, a random forest model, a decision tree classifier, a gradient boosted decision tree classifier, a neural network model, and/or any other suitable type of machine learning model, as aspects of the technology described herein are not limited in this respect. Example machine learning models and techniques for training such models are described herein including at least in the section “Machine Learning” and with respect to FIG. 1B, FIG. 1C, and FIG. 4.

In some embodiments, one or more machine learning models are trained to predict any suitable property or properties of the multi-chain protein, as aspects of the technology described herein are not limited in this respect. For example, the machine learning model(s) may be trained to predict one or more of the properties described herein including at least with respect to FIG. 1B. In some embodiments, the output of the trained machine learning model may be a binary indication as to whether or not the multi-chain protein has the particular property or properties. Additionally, or alternatively, the output may be indicative of the likelihood (e.g., a probability) that the multi-chain protein has the particular property or properties. Additionally, or alternatively, the output may indicate a value corresponding to the particular property or properties. For example, when the property is viscosity, the trained machine learning model(s) may output a value indicating the predicted viscosity of the multi-chain protein. Additionally, or alternatively, when the property is aggregation, the trained machine learning model(s) may output a value indicative aggregation (e.g., high molecular weight (HMW) percentage). Additionally, or alternatively, the output may be indicative of a degree to which the multi-chain protein has the property or properties. For example, a multi-chain protein may be predicted to have a “high,” “medium,” or “low” degree of the property (e.g., high aggregation, medium aggregation, low aggregation, etc.), a “slow” or “fast” degree of the property (e.g., slow pharmacokinetic clearance, fast pharmacokinetic clearance, etc.), or a “good” or “bad” degree of the property (e.g., good bioavailability, bad bioavailability, etc.). In some embodiments, the degree-based labels are determined based on threshold values corresponding to the particular property or properties. For example, a multi-chain protein predicted to have high aggregation may correspond to a protein with an HMW percentage of greater than or equal to 5%. A multi-chain protein predicted to have medium aggregation may correspond to a protein with an HMW % greater than or equal to 2% and less than 5%. A multi-chain protein predicted to have low aggregation may correspond to a protein with an HMW % less than 2%. Additionally, or alternatively, the output of the trained machine learning model may include any other suitable output, as aspects of the technology described herein are not limited in this respect.

It should be appreciated that illustrative process 300 may include one or more additional or alternative acts. For example, in some embodiments, illustrative process 300 includes all of the acts shown in FIG. 3. In some embodiments, illustrative process 300 includes acts 302-306 and act 310. In such embodiments, act 310 may include processing the numeric representation (e.g., obtained as a result of performing act 306) using the trained machine learning model(s). In some embodiments, illustrative process 300 further includes an act for manufacturing the multi-chain protein. In some embodiments, illustrative process 300 further includes an act of expressing (e.g., as part of phage display, using a cell-based expression system, or using a cell-free expression system) a fragment or all of the multi-chain protein and performing an assay (e.g., in vitro assay, phage display screening, viscosity assay) to confirm if the multi-chain protein has the one or more properties predicted by the machine learning model(s). If the multi-chain-protein is confirmed by results of the assay to have the one or more properties predicted by the machine learning model(s), then illustrative process 300 further includes an act of selecting the multi-chain protein for further testing (e.g., testing of its properties as a therapy). In some embodiments, illustrative process 300 further includes an act of producing (e.g., harvesting or purifying protein synthesized by an expression system) a fragment or all of the multi-chain protein and performing an assay (e.g., phage display screening, viscosity assay, in vitro assay) to confirm if the multi-chain protein has the one or more properties predicted by the machine learning model(s). If the multi-chain protein is confirmed by results of the assay to have the one or more properties predicted by the machine learning model(s), then illustrative process 300 further includes an act of selecting the multi-chain protein for further testing (e.g., testing of its properties as a therapy).

FIG. 4 is a flowchart of an illustrative process 400 for training a machine learning model to predict one or more properties of a multi-chain protein, according to some embodiments of the technology described herein. One or more (e.g., all) of the acts of process 400 may be performed automatically by any suitable computing device(s). For example, the act(s) may be performed by a laptop computer, a desktop computer, one or more servers, in a cloud computing environment, computing device(s) 250 as described herein including at least with respect to FIG. 2, computing system 700 as described herein including at least with respect to FIG. 7, and/or in any other suitable way, as aspects of the technology described herein are not limited in this respect.

At act 402, training data is generated. As shown in FIG. 4, act 402 include acts 412, 414, 416, and/or act 418. In some embodiments, act 402 includes one or more additional or alternative acts than those shown in FIG. 4. For example, act 402 may include all of acts 412, 414, 416, and 418. As another example, act 402 may include acts 412-416.

At act 412, initial data is obtained for a plurality of multi-chain proteins, each of which includes at least two chains. In some embodiments, the initial data is obtained from one or more user(s) (e.g., the user(s) uploading the initial data). In some embodiments, the initial data is obtained from a data store (e.g., multi-chain protein data store 210 shown in FIG. 2).

The initial data may include data for any suitable number of multi-chain proteins, as aspects of the technology described herein are not limited in this respect. For example, the initial data may include data for at least 100, at least 125, at least 150, at least 175, at least 200, at least 225, at least 250, at least 260, at least 280, at least 290, at least 300, at least 310, at least 320, at least 330, at least 340, at least 350, at least 375, at least 400, at least 450, at least 500, at least 600, at least 700, at least 800, at least 900, at least 1,000, or at least any other suitable number of multi-chain proteins. In some embodiments, the initial data includes data for at most 5,000, at most 2,500, at most 1,000, at most 900, at most 800, at most 800, at most 700, at most 600, at most 500, at most 450, at most 400, at most 375, at most 350, at most 340, at most 330, at most 320, at most 310, at most 300, at most 290, at most 280, at most 270, at most 260, at most 250, or at most any other suitable number of multi-chain proteins. It should be appreciated that any of the above-listed upper bounds may be coupled with any of the above-listed lower bounds. For example, the initial data may include data for a number of multi-chain proteins between 100 and 5,000, between 150 and 2,500, between 200, and 1,000, between 250 and 500, between 280 and 300, or a number of multi-chain proteins within any other suitable range.

In some embodiments, the initial data indicates, for each particular multi-chain protein, (i) one or more properties of the particular multi-chain protein and (ii) sequence data for the particular multi-chain protein.

In some embodiments, the one or more properties indicated by the initial data are the one or more properties that the machine learning model is trained to predict at act 404 of process 400. For example, if the machine learning model is trained to predict the viscosity of a multi-chain protein, then the initial data may indicate a measured viscosity of each multi-chain protein. As another example, if the machine learning model is trained to predict the aggregation of a multi-chain protein, then the initial data may indicate a measured aggregation of each multi-chain protein.

In some embodiments, the sequence data for a particular multi-chain protein indicates a respective amino acid sequence specifying at least a portion (e.g., all) of each chain of the particular multi-chain protein. For example, if a multi-chain protein includes a first chain and a second chain, then the sequence data for the particular multi-chain protein may indicate a first amino acid sequence specifying at least a portion (e.g., all) of the first chain and second amino acid sequence specifying at least a portion (e.g., all) of the second chain. In some embodiments, the sequence data indicates, for each particular chain of the multi-chain protein, a respective nucleic acid sequence corresponding to an amino acid sequence specifying at least a portion (e.g., all) of the particular chain. While embodiments of the technology described herein refer to using amino acid sequences (e.g., processing amino acid sequences using one or more machine learning models), it should be appreciated that nucleic acid sequence(s) may be used instead of amino acid sequence(s).

At act 414, the initial data is augmented to obtain augmented data. In some embodiments, the augmenting includes performing, for each particular multi-chain protein, act 414-1 and/or act 414-2.

At act 414-1, a respective concatenated amino acid sequence is generated for the particular multi-chain protein at least in part by concatenating a linker (e.g., amino acid sequence, one or more tokens) and the respective amino acid sequences indicated for the at least two chains of the particular multi-chain protein. Techniques for generating a concatenated amino acid sequence are described herein including at least with respect to act 304 of process 300 shown in FIG. 3. In some embodiments, act 414-1 includes generating the concatenated amino acid sequence without the linker. In such embodiments, generating the concatenated amino acid sequence includes concatenating the amino acid sequences specifying the portions of the chains of the particular multi-chain protein.

At act 414-2, permutations of the respective amino acid sequences indicated for the chains of the particular multi-chain protein are generated. In some embodiments, a permutation of the respective amino acid sequences includes an arrangement of the respective amino acid sequences within the concatenated amino acid sequence. In some embodiments, at least some (e.g., all) of the possible permutations may be generated for a particular multi-chain protein. Table 2 lists example permutations for a multi-chain protein having two chains. In Table 2, “A” refers to the amino acid sequence specifying at least a portion of the first chain, “B” refers to the amino acid sequence specifying at least a portion of the second chain, and “L” refers to linkers. An example of a multi-chain protein having two chains is an antibody with an immunoglobulin G (IgG) antibody structure having two light chains that are identical and two heavy chains that are identical. Here, “A” may refer to the amino acid sequence specifying at least a portion of the heavy chain and “B” may refer to the amino acid sequence specifying at least a portion of the light chain. Table 3 lists example permutations for a multi-chain protein having three chains. In Table 3, “A” refers to the amino acid sequence specifying at least a portion of the first chain, “B” refers to the amino acid sequence specifying at least a portion of the second chain, “C” refers to the amino acid sequence specifying at least a portion of the third chain, and “L” refers to linkers. An example of a multi-chain protein having three chains is a bispecific antibody with a Y-shaped structure having a light chain and a heavy chain forming one arm and only a heavy chain forming the other arm, where the two heavy chains are different amino acid sequences. Here, “A” may refer to the amino acid sequence specifying at least a portion of the first heavy chain, “B” may refer to the amino acid sequence specifying at least a portion of the second heavy chain, and “C” may refer to the amino acid sequence specifying at least a portion of the light chain. Table 4 lists example permutations for a multi-chain protein having four chains. An example of a multi-chain protein with four chains is a bispecific antibody with an immunoglobulin G (IgG) antibody structure having two different heavy chains and two different light chains. In Table 4, “A1” refers to the amino acid sequence specifying at least a portion of the first chain, “A2” refers to the amino acid sequence specifying at least a portion of the second chain, “B1” refers to the amino acid sequence specifying at least a portion of the third chain, and “B2” refers to the amino acid sequence specifying at least a portion of the fourth chain. In the context of a bispecific antibody having two different heavy chains and two different light chains, “A1” may refer to the amino acid sequence specifying at least a portion of the first heavy chain, “A2” may refer to the amino acid sequence specifying at least a portion of the second heavy chain, “B1” may refer to the amino acid sequence specifying at least a portion of the first light chain, and “B2” may refer to the amino acid sequence specifying at least a portion of the second light chain. While Tables 2, 3, and 4 show example permutations for multi-chain proteins having two, three, and four chains, respectively, it should be appreciated that act 414-2 may apply to multi-chain proteins having any suitable number of chains. It should also be appreciated that, though not shown, the concatenated sequences shown in Tables 2, 3, and 4 may exclude the linker.

TABLE 2
Permutations for a multi-chain protein having two chains.

| Permutation # | Concatenated Sequence |
|---|---|
| Permutation 1 | A-L-B |
| Permutation 2 | B-L-A |

TABLE 3
Permutations for a multi-chain protein having three chains.

| Permutation # | Concatenated Sequence |
|---|---|
| Permutation 1 | A-L-B-L-C |
| Permutation 2 | A-L-C-L-B |
| Permutation 3 | B-L-A-L-C |
| Permutation 4 | B-L-C-L-A |
| Permutation 5 | C-L-A-L-B |
| Permutation 6 | C-L-B-L-A |

TABLE 4
Permutations for a multi-chain protein having four chains,
such as a bispecific antibody heaving two different
heavy chains and two different light chains.

| Permutation # | Concatenated Sequence |
|---|---|
| Permutation 1 | A1-L-B1-L-A2-L-B2 |
| Permutation 2 | A1-L-B1-L-B2-L-A2 |
| Permutation 3 | A1-L-A2-L-B1-L-B2 |
| Permutation 4 | A1-L-A2-L-B2-L-B1 |
| Permutation 5 | A1-L-B2-L-A2-L-B1 |
| Permutation 6 | A1-L-B2-L-B1-L-A2 |
| Permutation 7 | B1-L-A1-L-B2-L-A2 |
| Permutation 8 | B1-L-A1-L-A2-L-B2 |
| Permutation 9 | B1-L-A2-L-B2-L-A1 |
| Permutation 10 | B1-L-A2-L-A1-L-B2 |
| Permutation 11 | B1-L-B2-L-A1-L-A2 |
| Permutation 12 | B1-L-B2-L-A2-L-A1 |
| Permutation 13 | A2-L-A1-L-B1-L-B2 |
| Permutation 14 | A2-L-A1-L-B2-L-B1 |
| Permutation 15 | A2-L-B1-L-A1-L-B2 |
| Permutation 16 | A2-L-B1-L-B2-L-A1 |
| Permutation 17 | A2-L-B2-L-A1-L-B1 |
| Permutation 18 | A2-L-B2-L-B1-L-A1 |
| Permutation 19 | B2-L-A1-L-A2-L-B1 |
| Permutation 20 | B2-L-A1-L-B1-L-A2 |
| Permutation 21 | B2-L-A2-L-B1-L-A1 |
| Permutation 22 | B2-L-A2-L-A1-L-B1 |
| Permutation 23 | B2-L-B1-L-A1-L-A2 |
| Permutation 24 | B2-L-B1-L-A2-L-A1 |

At act 416, the augmented data is encoded to obtain numeric representations of the augmented data. In some embodiments, encoding the augmented data includes encoding the concatenated amino acid sequences generated at act 414 to obtain numeric representations of the concatenated amino acid sequences. In some embodiments, this includes encoding the permutations generated at act 414-2. Techniques for encoding concatenated amino acid sequences are described herein including at least with respect to act 306 of process 300 shown in FIG. 3.

At act 418, a dimensionality of each of the numeric representations is reduced to obtain training data. Techniques for reducing a dimensionality of a numeric representation are described herein including at least with respect to act 308 of process 300 shown in FIG. 3.

At act 404, a machine learning model is trained, using the training data, to predict one or more properties of the multi-chain protein thereby obtaining parameter values for the trained machine learning model. As described herein including at least with respect to act 310 of process 300 shown in FIG. 3, the machine learning model may include any suitable machine learning model for predicting one or more properties of a multi-chain protein, as aspects of the technology described herein are not limited in this respect. In some embodiments, the machine learning model may be trained using any suitable training technique(s), including supervised techniques, semi-supervised techniques, unsupervised techniques, or any suitable combination thereof as aspects of the technology described herein are not limited in this respect. As one example, in the supervised training context, the reduced-dimension representation of a concatenated sequence generated for a multi-chain protein may be provided as input to the machine learning model, which may output one or more predicted properties of the multi-chain protein. Differences between the one or more predicted properties and the one or more known properties of the multi-chain protein (e.g., the one or more properties obtained at act 412) may be used to determine and update the parameter values of the machine learning model. At act 406, the parameter values of the machine learning model are stored. For example, the parameter values of the machine learning model may be stored in a machine learning model data store such as the machine learning model data store 220 shown in FIG. 2. To use the machine learning model (e.g., at act 310 of process 300 shown in FIG. 3), the stored parameter values may be accessed.

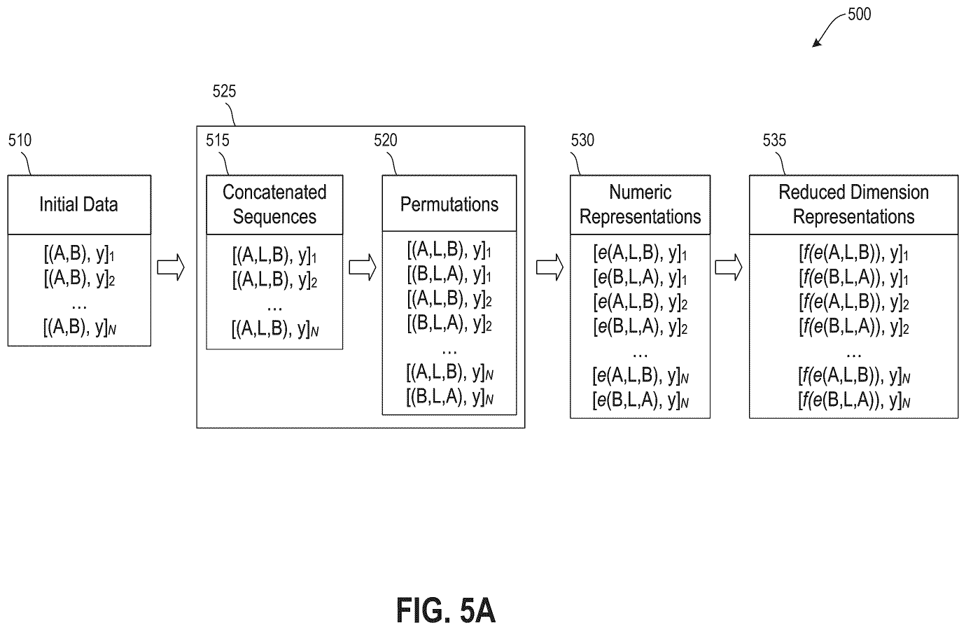

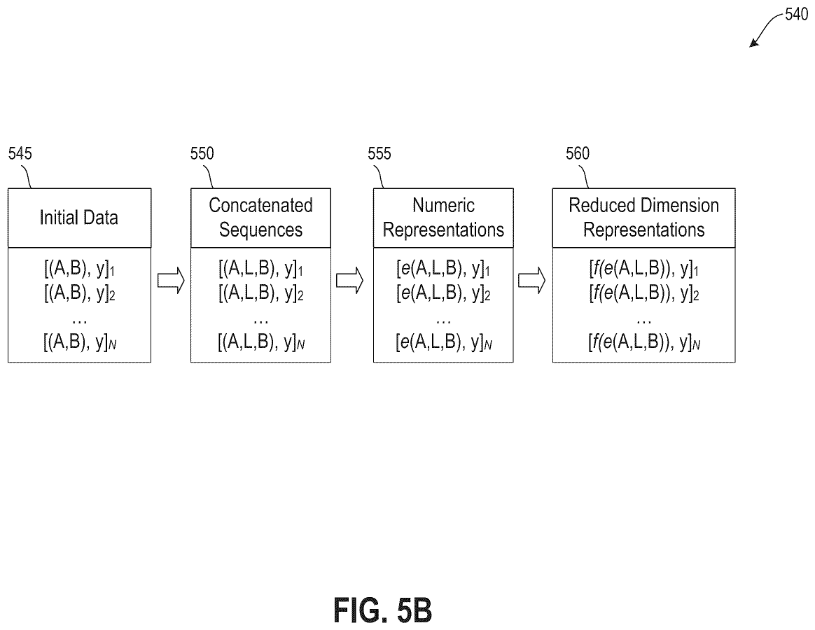

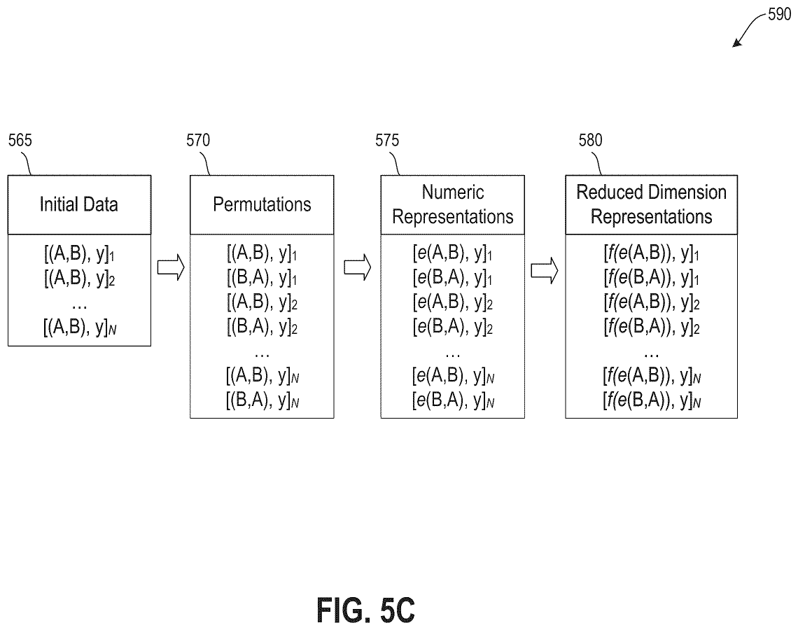

FIG. 5A is an illustrative example 500 of generating training data used for training a machine learning model to predict one or more properties of a multi-chain protein, according to some embodiments of the technology described herein. The illustrative example 500 is an example implementation of act 402 of process 400 shown in FIG. 4.

As shown in FIG. 5A, initial data 510 is obtained for a set of N multi-chain proteins, where N is any suitable number. The set of N multi-chain proteins includes a first multi-chain protein, a second multi-chain protein, and an Nth multi-chain protein. Each of the multi-chain proteins includes two chains. The initial data includes, for each of the multi-chain proteins, sequence data and one or more properties (“y”) of the multi-chain protein. The sequence data includes, for each particular multi-chain protein, an amino acid sequence (“A”) specifying at least a portion of the first chain of the particular multi-chain protein and an amino acid sequence (“B”) specifying at least a portion of the second chain of the particular multi-chain protein. Techniques for obtaining initial data for a plurality of multi-chain proteins are described herein including at least with respect to act 412 of process 400 shown in FIG. 4.

As shown in FIG. 5A, the initial data 510 is augmented to obtain augmented data 525. Augmenting the data includes generating concatenated sequences 515. The concatenated sequences include linkers (“L”) concatenated between the amino acid sequences specifying the chains of each multi-chain protein. Techniques for generating concatenated sequences are described herein including at least with respect to act 414-1 of process 400 shown in FIG. 4.

Augmenting the data also includes generating permutations 520 for each multi-chain protein. This includes generating different arrangements of the amino acid sequences specifying the chains of each multi-chain protein. For example, as shown in FIG. 5A, the first permutation for the first multi-chain protein includes the amino acid sequence “A” followed by linker “L” followed by the amino acid sequence “B”. The second permutation for the first multi-chain protein includes the amino acid sequence “B” followed by linker “L” followed by amino acid sequence “A”. The one or more properties obtained for a particular multi-chain protein are paired with each permutation generated for that particular multi-chain protein. Techniques for generating permutations for multi-chain proteins are described herein including at least with respect to act 414-2 of process 400 shown in FIG. 4.

Augmented data 525 is encoded to obtain the numeric representations 530. In particular, each of the permutations 520 (e.g., concatenated sequences) is encoded to obtain numeric representations of the permutations. Techniques for encoding augmented data are described herein including at least with respect to act 416 of process 400 shown in FIG. 4.

A dimensionality of each of the numeric representations 530 is reduced to obtain reduced-dimension representations 535. Techniques for reducing the dimensions of a numeric representation of a concatenated sequence are described herein including at least with respect to act 418 of process 400 shown in FIG. 4. In some embodiments, the reduced-dimension representations 535 are used to train one or more machine learning models to predict one or more properties of a multi-chain protein. In some embodiments, the act of reducing the dimensionality of numeric representations 530 is optional. In such embodiments, the numeric representations 530 may be used to train the one or more machine learning models to predict the one or more properties of a multi-chain protein.

FIG. 5B is an illustrative example 540 of generating training data used for training a machine learning model to predict one or more properties of a multi-chain protein, according to some embodiments of the technology described herein. The illustrative example 540 is an example implementation of act 402 of process 400 shown in FIG. 4.

As shown in FIG. 5B, initial data 545 is obtained for a set of N multi-chain proteins, where N is any suitable number. The set of N multi-chain proteins includes a first multi-chain protein, a second multi-chain protein, and an Nth multi-chain protein. Each of the multi-chain proteins includes two chains. The initial data includes, for each of the multi-chain proteins, sequence data and one or more properties (“y”) of the multi-chain protein. The sequence data includes, for each particular multi-chain protein, an amino acid sequence (“A”) specifying at least a portion of the first chain of the particular multi-chain protein and an amino acid sequence (“B”) specifying at least a portion of the second chain of the particular multi-chain protein. Techniques for obtaining initial data for a plurality of multi-chain proteins are described herein including at least with respect to act 412 of process 400 shown in FIG. 4.

As shown in FIG. 5B, the initial data 545 is augmented by generating concatenated sequences 550. The concatenated sequences include linkers (“L”) concatenated between the amino acid sequences specifying the chains of each multi-chain protein. Techniques for generating concatenated sequences are described herein including at least with respect to act 414-1 of process 400 shown in FIG. 4.

Concatenated sequences 550 are encoded to obtain the numeric representations 555 of the concatenated sequences 550. In particular, each of the concatenated sequences 550 is encoded to obtain numeric representations of the permutations. Techniques for encoding concatenated sequences are described herein including at least with respect to act 416 of process 400 shown in FIG. 4.

A dimensionality of each of the numeric representations 555 is reduced to obtain reduced-dimension representations 560. Techniques for reducing the dimensions of a numeric representation of a concatenated sequence are described herein including at least with respect to act 418 of process 400 shown in FIG. 4. In some embodiments, the reduced-dimension representations 560 are used to train one or more machine learning models to predict one or more properties of a multi-chain protein. In some embodiments, the act of reducing the dimensionality of numeric representations 555 is optional. In such embodiments, the numeric representations 555 may be used to train the one or more machine learning models to predict the one or more properties of a multi-chain protein.

FIG. 5C is an illustrative example 590 of generating training data used for training a machine learning model to predict one or more properties of a multi-chain protein, according to some embodiments of the technology described herein. The illustrative example 590 is an example implementation of act 402 of process 400 shown in FIG. 4.

As shown in FIG. 5C, initial data 565 is obtained for a set of N multi-chain proteins, where N is any suitable number. The set of N multi-chain proteins includes a first multi-chain protein, a second multi-chain protein, and an Nth multi-chain protein. Each of the multi-chain proteins includes two chains. The initial data includes, for each of the multi-chain proteins, sequence data and one or more properties (“y”) of the multi-chain protein. The sequence data includes, for each particular multi-chain protein, an amino acid sequence (“A”) specifying at least a portion of the first chain of the particular multi-chain protein and an amino acid sequence (“B”) specifying at least a portion of the second chain of the particular multi-chain protein. Techniques for obtaining initial data for a plurality of multi-chain proteins are described herein including at least with respect to act 412 of process 400 shown in FIG. 4.

As shown in FIG. 5C, the initial data 565 is augmented by generating permutations 570 for each multi-chain protein. This includes generating different arrangements of the amino acid sequences specifying the chains of each multi-chain protein. For example, as shown in FIG. 5C, the first permutation for the first multi-chain protein includes the amino acid sequence “A” followed by the amino acid sequence “B”. The second permutation for the first multi-chain protein includes the amino acid sequence “B” followed by amino acid sequence “A”. The one or more properties obtained for a particular multi-chain protein are paired with each permutation generated for that particular multi-chain protein. Techniques for generating permutations for multi-chain proteins are described herein including at least with respect to act 414-2 of process 400 shown in FIG. 4.

The permutations 570 are encoded to obtain the numeric representations 575 of the permutations 570. In particular, each of the permutations 570 is encoded to obtain numeric representations of the permutations. Techniques for encoding augmented data are described herein including at least with respect to act 416 of process 400 shown in FIG. 4.

A dimensionality of each of the numeric representations 575 is reduced to obtain reduced-dimension representations 580. Techniques for reducing the dimensions of a numeric representation of a concatenated sequence are described herein including at least with respect to act 418 of process 400 shown in FIG. 4. In some embodiments, the reduced-dimension representations 580 are used to train one or more machine learning models to predict one or more properties of a multi-chain protein. In some embodiments, the act of reducing the dimensionality of numeric representations 575 is optional. In such embodiments, the numeric representations 575 may be used to train the one or more machine learning models to predict the one or more properties of a multi-chain protein.

### Example

This example shows that the techniques developed by the inventors for training and using a machine learning model to predict aggregation of multi-chain proteins are an improvement over conventional techniques for training and using a machine learning model to predict aggregation of multi-chain proteins. This example includes the following sections: “Dataset,” “Training Data Augmentation,” “Encoding,” “Machine Learning Model Training,” and “Machine Learning Model Performance.”

### Dataset

A dataset was used to train and test the performance of a machine learning model trained to predict protein aggregation. The dataset includes, for each multi-chain protein in the dataset, amino acid sequence(s) of the multi-chain protein and a corresponding label indicative of aggregation of the multi-chain protein. In particular, one of three labels (e.g., high, medium, or low) was assigned to each protein in the dataset based on high-molecular-weight percentage (% HMW) measurements of the protein. % HMW thresholds used to assign the labels are shown in Table 5. Table 6 shows the number of multi-chain proteins in the dataset assigned to each label. The % HMW measurements of the proteins were obtained at 2-weeks of incubation and a temperature of 40° C. Column 1 of Table 6 includes only multi-chain proteins for which % HMW measurements were obtained at concentrations of less than 15 mg/mL. Column 2 includes all multi-chain proteins in the dataset including multi-chain proteins for which % HMW measurements were obtained at concentrations of greater than or equal to 15 mg/mL, as well as at concentrations less than 15 mg/mL.

TABLE 5
Dataset labels and aggregation thresholds.

| Label | Aggregation Threshold |
|---|---|
| Low | <2% HMW |
| Medium | ≥2% HMW and <5% HMW |
| High | ≥5% HMW |

TABLE 6
Number of proteins in dataset corresponding to each label.

| Label | # Proteins (<15 mg/mL) | # Proteins |
|---|---|---|
| Low (0) | 216 | 258 |
| Medium (1) | 93 | 93 |
| High (2) | 64 | 64 |

### Augmentation

The dataset was augmented prior to training. This included concatenating the amino acid sequences specifying each of the chains of the multi-chain protein. For example, if a multi-chain protein had two chains, the two amino acid sequences specifying the two chains were concatenated. For some of the multi-chain proteins, a linker (e.g., a linker of 90 mask tokens) was concatenated between the amino acid sequences specifying the chains. A linker was not added between the amino acid sequences if it would cause the total length of the concatenated sequence (e.g., the sequences+linker) to exceed the sequence length limit (e.g., 1022) imposed by ESM-1b.

The dataset was also augmented by generating permutations of the amino acid sequences within the concatenated sequences. Consider, for example, a multi-chain protein with (i) a first amino acid sequence, A, specifying a first chain of the multi-chain protein and (ii) a second amino acid sequence, B, specifying a second chain of the multi-chain protein. The permutations of the concatenated sequence (including a linker) would include: A-L-B and B-L-A.

The augmented data included 316 concatenated sequences. The number of concatenated sequences corresponding to each label are shown in Table 7.

TABLE 7
Number of sequences in augmented data
set corresponding to each label.

| Label | # Sequences |
|---|---|
| Low (0) | 240 |
| Medium (1) | 40 |
| High (2) | 36 |

### Encoding

Prior to training and testing the machine learning model, the augmented training data was encoded. A protein language model was used for encoding. In particular, the Evolutionary Scale Modeling 1b (ESM-1b) model was used. ESM-1b is described by described by Rives, A., et al. (“Biological structure and function emerge from scaling unsupervised learning to 250 million protein sequences.” Proceedings of the National Academy of Sciences 118.15 (2021): e2016239118.), which is incorporated by reference herein in its entirety. Table 8 lists details about ESM-1b.

TABLE 8
ESM-1b details.

| | | | | | Embedding |
|---|---|---|---|---|---|
| Shorthand | esm.pretrained. | # Layers | # Parameters | Dataset | Dimension |
| ESM-1b | esm1b_t33_650M_UR50S | 33 | 650 M | UR50/S 2018_03 | 1280 |

### Machine Learning Model Training

The augmented data (e.g., the 316 concatenated sequences) was divided into a training set (e.g., 70%) and test set (30%). In particular, in this example, the augmented data was divided in a group-based fashion. In other words, all permutations of a given multi-chain protein were always together in either the training set or the test set. This was done to ensure that no leakage occurred during evaluation. 100 training/test splits were performed. The distribution of performance across all splits was obtained.

The training set was fitted and transformed using three steps: (1) zero variance features were removed, (2) the data was standardized using a mean of 0 and standard deviation of 1, and (3) the dimensionality was reduced using principal components analysis (PCA). With respect to the dimensionality reduction step, the number of components which captured 95% of the variation was selected. The final trained machine learning model used 15 components.

After the training data was fitted and transformed, it was passed to a logistic regression model with the L2 regularization penalty. The logistic regression model was tuned using Grid Search and 3-fold group-based cross-validation. A range of values of C (e.g., the inverse of regularization strength) were explored (e.g., [0.003, 0.03, 0.3]), as well as the class weight parameter (e.g., [“balanced,” None]). The final machine learning model had C=0.3 and no class weight (class_weight=“None”).

### Machine Learning Model Performance

Different models were trained and tested to evaluate the effectiveness of the data augmentation techniques. In particular, six different models were trained and tested. The six models and their median performance metrics are shown in Table 9-1 and Table 9-2. The standard deviation for the performance metrics for three of the models are listed in Table 10. The six models include: the “Linker+All Permutations” model, the “Linker” model, the “All Permutations” model, the “Single Permutation” model, the “Average” model, and the “Weighted Average” model.

The “Linker+All Permutations” model was trained to predict aggregation of a multi-chain protein using training data that was generated by augmenting initial data obtained for multiple multi-chain proteins and encoding the augmented data. The augmenting included, for each of the multiple multi-chain proteins: (a) concatenating amino acid sequences specifying each of the chains of the multi-chain protein, (b) concatenating a linker between the amino acid sequences specifying the chains, and (c) generating all permutations of the amino acid sequences within the concatenated sequences.

The “Linker” model was trained to predict aggregation of a multi-chain protein using training data that was generated by augmenting initial data obtained for multiple multi-chain proteins and encoding the augmented data. The augmenting included, for each of the multiple multi-chain proteins: (a) concatenating amino acid sequences specifying each of the chains of the multi-chain protein, and (b) concatenating a linker between the amino acid sequences specifying the chains.

The “All Permutations” model was trained to predict aggregation of a multi-chain protein using training data that was generated by augmenting initial data obtained for multiple multi-chain proteins and encoding the augmented data. The augmenting included, for each of the multiple multi-chain proteins: (a) concatenating amino acid sequences specifying each of the chains of the multi-chain protein, and (b) generating all permutations of the amino acid sequences within the concatenated sequences.

The “Single Permutation” model was trained to predict aggregation of a multi-chain protein using training data that was generated by, for each of multiple multi-chain proteins: (a) concatenating amino acid sequences specifying each of the chains of the multi-chain protein, and (b) encoding the concatenated amino acid sequences.

The “Average” model was trained to predict aggregation of a multi-chain protein using training data that was generated by, for each of multiple multi-chain proteins: (a) separately encoding each amino acid sequence specifying each of the chains of the multi-chain protein to obtain a respective numeric representation of each amino acid sequence, and (b) averaging the numeric representations obtained for the amino acid sequences.

The “Weighted Average” model was trained to predict aggregation of a multi-chain protein using training data that was generated by, for each of multiple multi-chain proteins: (a) separately encoding each amino acid sequence specifying each of the chains of the multi-chain protein to obtain a respective numeric representation of each amino acid sequence, and (b) determining a weighted average of the numeric representations obtained for the amino acid sequences, where the weight assigned to a particular numeric representation is proportional to the length of the amino acid sequence for which the numeric representation was obtained. For example, if a first amino acid sequence specifying a first chain of the multi-chain protein is 50% longer than a second amino acid sequence specifying a second chain of the multi-chain protein, then a weight of 1.5 may be assigned to the numeric representation obtained for the first amino acid sequence when determining the weighted average.

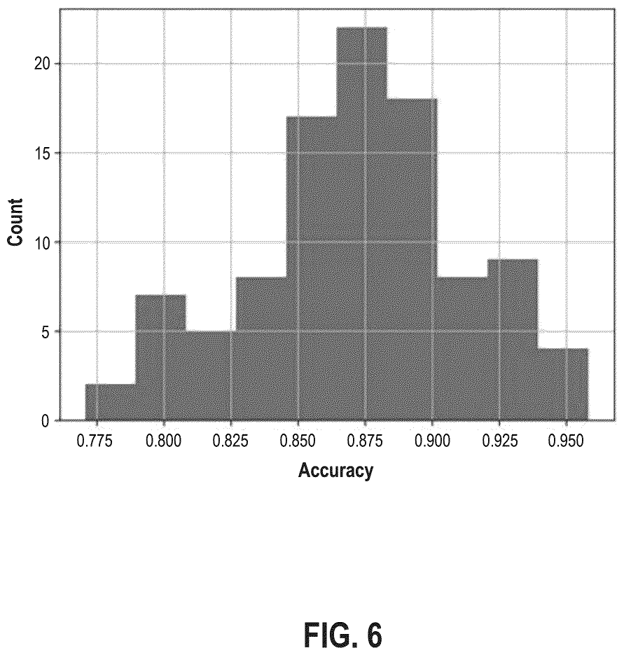

As shown in Table 9-1 and Table 9-2, the machine learning model trained on the data augmented with both the linker and permutations resulted in the highest performance. The distribution of accuracy of this model is shown in FIG. 6. In Tables 9-1 and 9-2, the “test statistic” is the sum of ranks of the differences above zero from the Wilcoxon signed-rank test.

TABLE 9-1
Median performance metrics.

| | Train: Matthews Correlation | Test: | | |
|---|---|---|---|---|
| Model | Coefficient (MCC) | MCC | Train: Accuracy | Test: Accuracy |
| Linker + All | 0.79 | 0.68 | 0.92 | 0.88 |
| Permutations | | | | |
| Linker | 0.75 | 0.66 | 0.90 | 0.86 |
| All Permutations | 0.71 | 0.59 | 0.89 | 0.84 |
| Single Permutation | 0.67 | 0.57 | 0.85 | 0.80 |
| Average | 0.59 | 0.49 | 0.78 | 0.72 |
| Weighted Average | 0.59 | 0.48 | 0.78 | 0.73 |

TABLE 9-2
Median performance metrics.

| | Train: Area Under the Receiver | | Test | |
|---|---|---|---|---|
| Model | Operating Characteristic Curve (AUC) | Test: AUC | Statistic | p-value |
| Linker + All | 0.97 | 0.92 | | |
| Permutations | | | | |
| Linker | 0.95 | 0.91 | 1939 | 0.043919 |
| All Permutations | 0.95 | 0.89 | 406 | 3.5E−12 |
| Single Permutation | 0.93 | 0.89 | 349 | 7.33E−14 |
| Average | 0.91 | 0.85 | 87 | 5.18E−17 |
| Weighted Average | 0.92 | 0.86 | 94 | 6.35E−17 |

TABLE 10
Standard deviation of the performance metrics.

| | Train: | Test: | Train: | Test: | Train: | Test: |
|---|---|---|---|---|---|---|
| Model | MCC | MCC | Accuracy | Accuracy | AUC | AUC |
| Linker + All Permutations | 0.06 | 0.09 | 0.02 | 0.04 | 0.01 | 0.05 |
| Linker | 0.06 | 0.12 | 0.03 | 0.06 | 0.02 | 0.06 |
| Average | 0.06 | 0.09 | 0.03 | 0.04 | 0.02 | 0.04 |

### Multi-Chain Proteins

Aspects of the disclosure relate to multi-chain proteins, which also may be referred to as protein complexes. As used herein, a “multi-chain protein” or “protein complex” refers to a group of two or more associated polypeptide chains (also referred to in some embodiments as “subunits”). A “polypeptide chain” generally refers to a linear, unbranched, series of amino acids (e.g., naturally occurring or non-naturally occurring) linked to one another by peptide bonds. A polypeptide chain may range in length from about 2 to about 30,000 amino acids in length, for example 2 to 100 amino acids, 10 to 50 amino acids, 5 to 1000 amino acids, 500 to 5000 amino acids, etc. In some embodiments, a polypeptide chain having a size of greater than 10 Daltons (Da) is referred to as a “protein”. “Associated polypeptide chains” refers to polypeptide chains that interact with one another to form a quaternary structure. In some embodiments, associated polypeptide chains are directly bound to one another, for example by covalent bonds, peptide bonds, etc. In some embodiments, associated polypeptide chains are not directly bound to one another, for example through non-covalent, protein-protein interactions. Multi-chain proteins may be obligate protein complexes or non-obligate protein complexes. An “obligate” protein complex requires the presence of one or more chaperone proteins to enable interaction between the peptide chains of the protein complex. Peptide chains of a “non-obligate” protein complex are capable of interacting to form a quaternary structure in the absence of chaperone proteins.

The number of chains in a multi-chain protein can vary. In some embodiments, a multi-chain protein comprises between 2 and 100 peptide chains (e.g., 2 and 100 subunits). In some embodiments, a multi-chain protein comprises between 2 and 10, 5 and 15, 10 and 20, 15 and 45, 25 and 50, 30 and 70, or 50 and 100, peptide chains. In some embodiments, a multi-chain protein comprises more than 100 (e.g., 120, 150, 200, etc.) peptide chains. In some embodiments, a multi-chain protein is a homomeric multi-chain protein (e.g., all subunits of the multi-chain protein are the same). In some embodiments, a multi-chain protein is a heteromeric multi-chain protein (e.g., the multi-chain protein comprises at least two different peptide chains).

Examples of multi-chain proteins include but are not limited to hemoglobin, insulin, transcription factor complexes, DNA polymerase, ribosomes, certain toxins, antibodies, G-protein-coupled receptors, and ion channels.

In some embodiments, a multi-chain protein is an antibody, also referred to as an “immunoglobulin”. An “antibody” is a protein complex typically comprising four peptide chains which form a “Y-shaped” structure and function to identify and neutralize objects in a subject (e.g., a mammalian subject, such as a human or mouse). Antibodies typically comprise two “light chains” and two “heavy chains” where a pair of light and heavy chains form an “arm” of the Y-shaped structure of the antibody. In some embodiments, an antibody light chain comprises a variable domain and a constant domain and is approximately 200-250 amino acids in length. In some embodiments, an antibody heavy chain comprises a variable domain and a constant domain, and is selected from an IgA, IgD, IgE, IgG, and IgM heavy chain. In some embodiments, an antibody heavy chain comprises between 450 and 550 amino acids. Antibodies typically neutralize foreign objects by binding to one or more antigens. In some embodiments, an antibody binds to an antigen selected from a bacterial antigen, viral antigen, parasitic antigen, host immune protein antigen, or cancer-associated antigen. Examples of bacterial antigens include but are not limited to bacterial surface proteins, lipopolysaccharides, and peptidoglycans. Examples of viral antigens include but are not limited to HIV peptide antigens, herpesvirus peptide antigens, coronavirus peptide antigens, etc. Examples of parasitic antigens include but are not limited to malaria surface antigens, helminth peptide antigens, etc. Examples of host immune protein antigens include but are not limited to cytokines (e.g., IL-2, IL-10, TNF-alpha, etc.) and immunoregulatory molecules (e.g., PD-1, PD-1L, CTLA, etc.). Examples of cancer antigens include but are not limited to tumor-specific antigens, such as mesothelin, Claudin, prostate-specific antigen (PSA), etc. When used as a therapy, an antibody typically binds to a “target” biological molecule or structure to contribute to a therapeutic effect. Examples of targets that a therapeutic antibody may bind to include antigens (e.g., tumor-associated antigens), receptors (e.g., cell surface receptors), and other proteins (e.g., immune checkpoint proteins).

### Machine Learning

In some embodiments, the techniques developed by the inventors include using one or more trained machine learning models to predict one or more properties of a multi-chain protein. The machine learning model(s) may include a non-linear regression model (e.g., a logistic regression model), a linear regression model, a support vector machine, a Gaussian mixture model, a random forest model, a decision tree classifier, a gradient boosted decision tree classifier, a neural network model, and/or any other suitable type of machine learning model, as aspects of the technology described herein are not limited in this respect. In some embodiments, the machine learning model(s) may include an ensemble of machine learning models of any suitable type (the machine learning models part of the ensemble may be termed “weak learners”).

As described above, in some embodiments, the machine learning model(s) may be implemented as a decision tree classifier. Any suitable type of decision tree classifier may be used and may be trained using any suitable supervised decision tree learning technique. For example, the decision tree classifier may be trained by the iterative dichotomizer technique (e.g., the ID3 algorithm as described, for example, in Quinlan, J. R. 1986. Induction of Decision Trees. Mach. Learn. 1, 1 (March 1986), 81-106)), the C4.5 technique (e.g., as described, for example, in Quinlan, J. R. C4.5: Programs for Machine Learning. Morgan Kaufmann Publishers, 1993), the classification and regression tree (CART) technique (e.g., as described, for example, in Breiman, Leo; Friedman, J. H.; Olshen, R. A.; Stone, C. J. (1984). Classification and regression trees. Monterey, CA: Wadsworth & Brooks/Cole Advanced Books & Software). It should be appreciated that a decision tree classifier may be trained using any other suitable training method, as aspects of the technology described herein are not limited in this respect.

In some embodiments, a gradient-boosted decision tree classifier may be used. The gradient-boosted decision tree classifier may be an ensemble of multiple decision tree classifiers (sometimes called “weak learners”). The prediction (e.g., classification) generated by the gradient-boosted decision tree classifier is formed based on the predictions generated by the multiple decision trees part of the ensemble. The ensemble may be trained using an iterative optimization technique involving calculation of gradients of a loss function (hence the name “gradient” boosting). Any suitable supervised training algorithm may be applied to training a gradient-boosted decision tree classifier including, for example, any of the algorithms described in Hastie, T.; Tibshirani, R.; Friedman, J. H. (2009). “10. Boosting and Additive Trees”. The Elements of Statistical Learning (2nd ed.). New York: Springer. pp. 337-384. In some embodiments, the gradient-boosted decision tree classifier may be implemented using any suitable publicly available gradient boosting framework such as XGBoost (e.g., as described, for example, in Chen, T., & Guestrin, C. (2016). XGBoost: A Scalable Tree Boosting System. In Proceedings of the 22nd ACM SIGKDD International Conference on Knowledge Discovery and Data Mining (pp. 785-794). New York, NY, USA: ACM.). The XGBoost software may be obtained from http://xgboost.ai, for example). Another example framework that may be employed is LightGBM (e.g., as described, for example, in Ke, G., Meng, Q., Finley, T., Wang, T., Chen, W., Ma, W., . . . . Liu, T.-Y. (2017). Lightgbm: A highly efficient gradient boosting decision tree. Advances in Neural Information Processing Systems, 30, 3146-3154.). The LightGBM software may be obtained from https://lightgbm.readthedocs.io/, for example).

In some embodiments, a neural network classifier may be used. The neural network classifier may be trained using any suitable neural network optimization software. The optimization software may be configured to perform neural network training by gradient descent, stochastic gradient descent, or in any other suitable way. In some embodiments, the Adam optimizer (Kingma, D. and Ba, J. (2015) Adam: A Method for Stochastic Optimization. Proceedings of the 3rd International Conference on Learning Representations (ICLR 2015)) may be used.

In some embodiments, a support vector machine (SVM) may be used. The SVM may be implemented using any suitable techniques such as, for example, any of the techniques described by Cristianini, N., and Shawe-Taylor, J. (“An introduction to support vector machines and other kernel-based learning methods.” Cambridge university press, 2000.), which is incorporated by reference herein in its entirety.

In some embodiments, a Gaussian mixture model may be used. The Gaussian mixture model may be implemented using any suitable techniques such as, for example, any of the techniques described by Reynolds, D. (“Gaussian mixture models.” Encyclopedia of biometrics 741.659-663 (2009)), which is incorporated by reference herein in its entirety.

In some embodiments, a random forest model may be used. The random forest model may be implemented using any suitable techniques such as, for example, any of the techniques described by Biau, G. (“Analysis of a random forests model.” The Journal of Machine Learning Research 13.1 (2012): 1063-1095.), which is incorporated by reference herein in its entirety.

### Computer Implementation

An illustrative implementation of a computer system 700 that may be used in connection with any of the embodiments of the technology described herein (e.g., such as the processes of FIG. 3 and FIG. 4) is shown in FIG. 7. The computer system 700 includes one or more processors 710 and one or more articles of manufacture that comprise non-transitory computer-readable storage media (e.g., memory 720 and one or more non-volatile storage media 730). The processor 710 may control writing data to and reading data from the memory 720 and the non-volatile storage media 730 in any suitable manner, as the aspects of the technology described herein are not limited to any particular techniques for writing or reading data. To perform any of the functionality described herein, the processor 710 may execute one or more processor-executable instructions stored in one or more non-transitory computer-readable storage media (e.g., the memory 720), which may serve as non-transitory computer-readable storage media storing processor-executable instructions for execution by the processor 710.

Computing system 700 may include a network input/output (I/O) interface 740 via which the computing device may communicate with other computing devices. Such computing devices may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

Computing system 700 may also include one or more user I/O interfaces 750, via which the computing device may provide output to and receive input from a user. The user I/O interfaces may include devices such as a keyboard, a mouse, a microphone, a display device (e.g., a monitor or touch screen), speakers, a camera, and/or various other types of I/O devices.

Further, it should be appreciated that a computer may be embodied in any of a number of forms, such as a rack-mounted computer, a desktop computer, a laptop computer, or a tablet computer, as examples. Additionally, a computer may be embedded in a device not generally regarded as a computer but with suitable processing capabilities, including a Personal Digital Assistant (PDA), a smartphone, a tablet, or any other suitable portable or fixed electronic device.

The above-described embodiments can be implemented in any of numerous ways. For example, the embodiments may be implemented using hardware, software, or a combination thereof. When implemented in software, the software code can be executed on any suitable processor (e.g., a microprocessor) or collection of processors, whether provided in a single computing device or distributed among multiple computing devices. It should be appreciated that any component or collection of components that perform the functions described above can be generically considered as one or more controllers that control the above-described functions. The one or more controllers can be implemented in numerous ways, such as with dedicated hardware, or with general purpose hardware (e.g., one or more processors) that is programmed using microcode or software to perform the functions recited above.

In this respect, it should be appreciated that one implementation of the embodiments described herein comprises at least one computer-readable storage medium (e.g., RAM, ROM, EEPROM, flash memory or other memory technology, CD-ROM, digital versatile disks (DVD) or other optical disk storage, magnetic cassettes, magnetic tape, magnetic disk storage or other magnetic storage devices, or other tangible, non-transitory computer-readable storage medium) encoded with a computer program (i.e., a plurality of executable instructions) that, when executed on one or more processors, performs the above-described functions of one or more embodiments. The computer-readable medium may be transportable such that the program stored thereon can be loaded onto any computing device to implement aspects of the techniques described herein. In addition, it should be appreciated that the reference to a computer program which, when executed, performs any of the above-described functions, is not limited to an application program running on a host computer. Rather, the terms computer program and software are used herein in a generic sense to reference any type of computer code (e.g., application software, firmware, microcode, or any other form of computer instruction) that can be employed to program one or more processors to implement aspects of the techniques described herein.

The terms “program” or “software” are used herein in a generic sense to refer to any type of computer code or set of computer-executable instructions that can be employed to program a computer or other processor to implement various aspects as described above. Additionally, it should be appreciated that according to one aspect, one or more computer programs that when executed perform methods of the present disclosure need not reside on a single computer or processor but may be distributed in a modular fashion among a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

When implemented in software, the software code can be executed on any suitable processor or collection of processors, whether provided in a single computer or distributed among multiple computers.

The foregoing description of implementations provides illustration and description but is not intended to be exhaustive or to limit the implementations to the precise form disclosed. Modifications and variations are possible in light of the above teachings or may be acquired from practice of the implementations. In other implementations the methods depicted in these figures may include fewer operations, different operations, differently ordered operations, and/or additional operations. Further, non-dependent blocks may be performed in parallel.

It will be apparent that example aspects, as described above, may be implemented in many different forms of software, firmware, and hardware in the implementations illustrated in the figures.

Having thus described several aspects and embodiments of the technology set forth in the disclosure, it is to be appreciated that various alterations, modifications, and improvements will readily occur to those skilled in the art. Such alterations, modifications, and improvements are intended to be within the spirit and scope of the technology described herein. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein. Those skilled in the art will recognize or be able to ascertain using no more than routine experimentation many equivalents to the specific embodiments described herein. It is, therefore, to be understood that the foregoing embodiments are presented by way of example only and that, within the scope of the appended claims and equivalents thereto, inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as an example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as an example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

The terms “approximately,” “substantially,” and “about” may be used to mean within ±20% of a target value in some embodiments, within ±10% of a target value in some embodiments, within ±5% of a target value in some embodiments, within ±2% of a target value in some embodiments. The terms “approximately,” “substantially,” and “about” may include the target value.

1. A method of predicting one or more properties of a multi-chain protein, the multi-chain protein including at least a first chain and a second chain, the method comprising:
using at least one computer hardware processor to perform:
obtaining sequence data for the multi-chain protein, the sequence data indicating a first amino acid sequence specifying at least a portion of the first chain and a second amino acid sequence specifying at least a portion of the second chain;
generating a concatenated amino acid sequence by concatenating the first amino acid sequence, a linker, and the second amino acid sequence;
encoding the concatenated amino acid sequence to obtain a numeric representation of the concatenated amino acid sequence; and
processing the numeric representation of the concatenated amino acid sequence using a trained machine learning model to obtain an output indicative of the one or more properties of the multi-chain protein.

2. The method of claim 1, wherein processing the numeric representation of the concatenated amino acid sequence using the trained machine learning model to obtain the output indicative of the one or more properties of the multi-chain protein comprises processing the numeric representation of the concatenated amino acid sequence using the trained machine learning model to obtain an output indicative of a degree of aggregation.

3. The method of claim 1, wherein processing the numeric representation of the concatenated amino acid sequence using the trained machine learning model to obtain the output indicative of the one or more properties of the multi-chain protein comprises processing the numeric representation of the concatenated amino acid sequence using the trained machine learning model to obtain an output indicative of a viscosity of the multi-chain protein.

4. The method of claim 1, wherein processing the numeric representation of the concatenated amino acid sequence using the trained machine learning model to obtain the output indicative of the one or more properties of the multi-chain protein comprises processing the numeric representation of the concatenated amino acid sequence using the trained machine learning model to obtain an output indicative of a degree of stability of the multi-chain protein.

5. The method of claim 1, wherein processing the numeric representation of the concatenated amino acid sequence using the trained machine learning model to obtain the output indicative of the one or more properties of the multi-chain protein comprises processing the numeric representation of the concatenated amino acid sequence using the trained machine learning model to obtain an output indicative of a degree of bioavailability of the multi-chain protein.

6. The method of claim 1, wherein processing the numeric representation of the concatenated amino acid sequence using the trained machine learning model to obtain the output indicative of the one or more properties of the multi-chain protein comprises processing the numeric representation of the concatenated amino acid sequence using the trained machine learning model to obtain an output indicative of a degree of pharmacokinetic clearance of the multi-chain protein.

7. The method of claim 1, wherein processing the numeric representation of the concatenated amino acid sequence using the trained machine learning model to obtain the output indicative of the one or more properties of the multi-chain protein comprises processing the numeric representation of the concatenated amino acid sequence using the trained machine learning model to obtain an output indicative of a productivity of the multi-chain protein.

8. The method of claim 1, wherein processing the numeric representation of the concatenated amino acid sequence using the trained machine learning model to obtain the output indicative of the one or more properties of the multi-chain protein comprises processing the numeric representation of the concatenated amino acid sequence using the trained machine learning model to obtain an output indicative of a binding affinity of the multi-chain protein to a target.

9. The method of claim 1, further comprising:
reducing a dimensionality of the numeric representation of the concatenated amino acid sequence to obtain a reduced-dimension representation of the numeric representation, the reduced-dimension representation of the numeric representation having fewer dimensions than the numeric representation,
wherein processing the numeric representation using the trained machine learning model to obtain the output indicative of the one or more properties of the multi-chain protein comprises processing the reduced-dimension representation of the numeric representation using the trained machine learning model to obtain the output indicative of the one or more properties of the multi-chain protein.

10. The method of claim 9, wherein reducing the dimensionality of the numeric representation of the concatenated amino acid sequence comprises reducing the dimensionality of the numeric representation of the concatenated amino acid sequence using principal components analysis (PCA).

11. The method of claim 1,
wherein encoding the concatenated amino acid sequence to obtain the numeric representation of the concatenated amino acid sequence comprises encoding the concatenated amino acid sequence using a protein language model,
wherein processing the numeric representation of the concatenated amino acid sequence using the trained machine learning model to obtain the output indicative of the one or more properties of the multi-chain protein comprises processing the numeric representation of the concatenated amino acid sequence using a non-linear regression model.

12. (canceled)

13. The method of claim 1, wherein the linker comprises one or more mask tokens or is a poly-alanine linker.

14. (canceled)

15. The method of claim 1, wherein the trained machine learning model was trained at least in part by:
generating training data at least in part by:
obtaining initial data for a plurality of multi-chain proteins, each of the plurality of multi-chain proteins including at least two chains, wherein the initial data indicates, for each particular multi-chain protein of the plurality of multi-chain proteins, one or more properties of the particular multi-chain protein and sequence data that indicates a respective amino acid sequence for each of the at least two chains of the particular multi-chain protein;
augmenting the initial data to obtain augmented data, the augmenting comprising, for each particular multi-chain protein of the plurality of multi-chain proteins (i) generating a respective concatenated amino acid sequence for the particular multi-chain protein at least in part by concatenating a linker and the respective amino acid sequences indicated for the at least two chains of the particular multi-chain protein and/or (ii) generating permutations of the respective amino acid sequences indicated for the at least two chains of the particular multi-chain protein; and
encoding the augmented data to obtain the training data;
training the machine learning model using the generated training data to predict the one or more properties of the multi-chain protein thereby obtaining values for parameters of the trained machine learning model; and
storing the parameter values for the trained machine learning model.

16. (canceled)

17. (canceled)

18. The method of claim 1, further comprising:
modifying, based on the output indicative of the one or more properties of the multi-chain protein, one or more residues of the first amino acid sequence and/or one or more residues of the second amino acid sequence.

19. The method of claim 1, further comprising:
expressing, based on the output indicative of the one or more properties of the multi-chain protein, the multi-chain protein or a fragment of the multi-chain protein to confirm if the multi-chain protein has the one or more properties by performing an assay; and
selecting, if results of the assay confirm the multi-chain protein has the one or more properties, the multi-chain protein for additional testing as a potential therapy.

20. A system, comprising:
at least one computer hardware processor; and
at least one non-transitory computer-readable storage medium storing processor-executable instructions that, when executed by the at least one computer hardware processor, cause the at least one computer hardware processor to perform a method of predicting one or more properties of a multi-chain protein, the multi-chain protein including at least a first chain and a second chain, the method comprising:
obtaining sequence data for the multi-chain protein, the sequence data indicating a first amino acid sequence specifying at least a portion of the first chain and a second amino acid sequence specifying at least a portion of the second chain;
generating a concatenated amino acid sequence by concatenating the first amino acid sequence, a linker, and the second amino acid sequence;
encoding the concatenated amino acid sequence to obtain a numeric representation of the concatenated amino acid sequence; and
processing the numeric representation of the concatenated amino acid sequence using a trained machine learning model to obtain an output indicative of the one or more properties of the multi-chain protein.

21. At least one non-transitory computer-readable storage medium storing processor-executable instructions that, when executed by at least one computer hardware processor, cause the at least one computer hardware processor to perform a method of predicting one or more properties of a multi-chain protein, the multi-chain protein including at least a first chain and a second chain, the method comprising:
obtaining sequence data for the multi-chain protein, the sequence data indicating a first amino acid sequence specifying at least a portion of the first chain and a second amino acid sequence specifying at least a portion of the second chain;
generating a concatenated amino acid sequence by concatenating the first amino acid sequence, a linker, and the second amino acid sequence;
encoding the concatenated amino acid sequence to obtain a numeric representation of the concatenated amino acid sequence; and
processing the numeric representation of the concatenated amino acid sequence using a trained machine learning model to obtain an output indicative of the one or more properties of the multi-chain protein.

22-36. (canceled)

37. The system of claim 20, wherein processing the numeric representation of the concatenated amino acid sequence using the trained machine learning model to obtain the output indicative of the one or more properties of the multi-chain protein comprises processing the numeric representation of the concatenated amino acid sequence using the trained machine learning model to obtain an output indicative of a degree of aggregation, a viscosity of the multi-chain protein, a degree of stability of the multi-chain protein, a degree of bioavailability of the multi-chain protein, a degree of pharmacokinetic clearance of the multi-chain protein, a productivity of the multi-chain protein, or a binding affinity of the multi-chain protein to a target.

38. The system of claim 20, where the method further comprises:
reducing a dimensionality of the numeric representation of the concatenated amino acid sequence to obtain a reduced-dimension representation of the numeric representation, the reduced-dimension representation of the numeric representation having fewer dimensions than the numeric representation,
wherein processing the numeric representation using the trained machine learning model to obtain the output indicative of the one or more properties of the multi-chain protein comprises processing the reduced-dimension representation of the numeric representation using the trained machine learning model to obtain the output indicative of the one or more properties of the multi-chain protein.

39. The system of claim 20,
wherein encoding the concatenated amino acid sequence to obtain the numeric representation of the concatenated amino acid sequence comprises encoding the concatenated amino acid sequence using a protein language model, and
wherein processing the numeric representation of the concatenated amino acid sequence using the trained machine learning model to obtain the output indicative of the one or more properties of the multi-chain protein comprises processing the numeric representation of the concatenated amino acid sequence using a non-linear regression model.

40. The system of claim 20, wherein the linker comprises one or more mask tokens or is a poly-alanine linker.

41. The system of claim 20, wherein the trained machine learning model was trained at least in part by:
generating training data at least in part by:
obtaining initial data for a plurality of multi-chain proteins, each of the plurality of multi-chain proteins including at least two chains, wherein the initial data indicates, for each particular multi-chain protein of the plurality of multi-chain proteins, one or more properties of the particular multi-chain protein and sequence data that indicates a respective amino acid sequence for each of the at least two chains of the particular multi-chain protein;
augmenting the initial data to obtain augmented data, the augmenting comprising, for each particular multi-chain protein of the plurality of multi-chain proteins (i) generating a respective concatenated amino acid sequence for the particular multi-chain protein at least in part by concatenating a linker and the respective amino acid sequences indicated for the at least two chains of the particular multi-chain protein and/or (ii) generating permutations of the respective amino acid sequences indicated for the at least two chains of the particular multi-chain protein; and
encoding the augmented data to obtain the training data;
training the machine learning model using the generated training data to predict the one or more properties of the multi-chain protein thereby obtaining values for parameters of the trained machine learning model; and
storing the parameter values for the trained machine learning model.

42. The at least one non-transitory computer-readable storage medium of claim 21, wherein processing the numeric representation of the concatenated amino acid sequence using the trained machine learning model to obtain the output indicative of the one or more properties of the multi-chain protein comprises processing the numeric representation of the concatenated amino acid sequence using the trained machine learning model to obtain an output indicative of a degree of aggregation, a viscosity of the multi-chain protein, a degree of stability of the multi-chain protein, a degree of bioavailability of the multi-chain protein, a degree of pharmacokinetic clearance of the multi-chain protein, a productivity of the multi-chain protein, or a binding affinity of the multi-chain protein to a target.

43. The at least one non-transitory computer-readable storage medium of claim 21, where the method further comprises:
reducing a dimensionality of the numeric representation of the concatenated amino acid sequence to obtain a reduced-dimension representation of the numeric representation, the reduced-dimension representation of the numeric representation having fewer dimensions than the numeric representation,
wherein processing the numeric representation using the trained machine learning model to obtain the output indicative of the one or more properties of the multi-chain protein comprises processing the reduced-dimension representation of the numeric representation using the trained machine learning model to obtain the output indicative of the one or more properties of the multi-chain protein.

44. The at least one non-transitory computer-readable storage medium of claim 21,
wherein encoding the concatenated amino acid sequence to obtain the numeric representation of the concatenated amino acid sequence comprises encoding the concatenated amino acid sequence using a protein language model, and
wherein processing the numeric representation of the concatenated amino acid sequence using the trained machine learning model to obtain the output indicative of the one or more properties of the multi-chain protein comprises processing the numeric representation of the concatenated amino acid sequence using a non-linear regression model.

45. The at least one non-transitory computer-readable storage medium of claim 21, wherein the linker comprises one or more mask tokens or is a poly-alanine linker.

46. The at least one non-transitory computer-readable storage medium of claim 21, wherein the trained machine learning model was trained at least in part by:
generating training data at least in part by:
obtaining initial data for a plurality of multi-chain proteins, each of the plurality of multi-chain proteins including at least two chains, wherein the initial data indicates, for each particular multi-chain protein of the plurality of multi-chain proteins, one or more properties of the particular multi-chain protein and sequence data that indicates a respective amino acid sequence for each of the at least two chains of the particular multi-chain protein;
augmenting the initial data to obtain augmented data, the augmenting comprising, for each particular multi-chain protein of the plurality of multi-chain proteins (i) generating a respective concatenated amino acid sequence for the particular multi-chain protein at least in part by concatenating a linker and the respective amino acid sequences indicated for the at least two chains of the particular multi-chain protein and/or (ii) generating permutations of the respective amino acid sequences indicated for the at least two chains of the particular multi-chain protein; and
encoding the augmented data to obtain the training data;
training the machine learning model using the generated training data to predict the one or more properties of the multi-chain protein thereby obtaining values for parameters of the trained machine learning model; and
storing the parameter values for the trained machine learning model.
